(19) United States
(12) Patent Application Publication
(10) Pub. No.: US 2024/0158377 A1
(43) Pub. Date: May 16, 2024

(54) NOVEL SALT FORMS OF A 4H-PYRAN-4-ONE STRUCTURED CYP11A1 INHIBITOR

(71) Applicant: Orion Corporation, Espoo (FI)

(72) Inventors: Oskari Karjalainen, Espoo (FI); Mikko Mäkelä, Espoo (FI); Mihaela Pop, Amsterdam (NL); Petteri Rummakko, Espoo (FI); Anna Shevchenko, Horsholm (DK); Kai Sinervo, Espoo (FI); Eija Tiainen, Espoo (FI)

(73) Assignee: Orion Corporation, Espoo (FI)

(21) Appl. No.: 18/548,487

(22) PCT Filed: Feb. 28, 2022
(86) PCT No.: PCT/FI2022/050130

§ 371 Date: Aug. 30, 2023

(30) Foreign Application Priority Data
Mar. 1, 2021 (FI) 20215217

(51) Int. Cl. C07D 405/14; A61K 9/20; A61K 31/454

(52) U.S. Cl. CPC C07D 405/14; A61K 9/20; A61K 31/454

(57) ABSTRACT

The present invention relates to novel salts, particularly crystalline salts, of 2-(isoindolin-2-ylmethyl)-5-((1-(methyl sulfonyl)piperidin-4-yl)methoxy)-4H-pyran-4-one (I) which are particularly suitable for use in the manufacture of pharmaceutical 5 compositions. Furthermore, the invention relates to pharmaceutical compositions comprising such novel salts. Compound (I) is a selective inhibitor of CYP11A1 enzyme and is useful in the treatment of hormonally regulated cancers, such as prostate cancer and breast cancer.

### TECHNICAL FIELD

The present invention relates to novel salts of 2-(isoindolin-2-ylmethyl)-5-((1-(methylsulfonyl)piperidin-4-yl)methoxy)-4H-pyran-4-one (I) and to preparation thereof. Furthermore, the invention relates to pharmaceutical compositions comprising such novel salts.

### BACKGROUND OF THE INVENTION

The compound 2-(isoindolin-2-ylmethyl)-5-((1-(methylsulfonyl)piperidin-4-yl)methoxy)-4H-pyran-4-one of formula (I) and derivatives thereof have been disclosed in WO 2018/115591. Compound of formula (I) is a selective inhibitor of CYP11A1 enzyme and is useful in the treatment of hormonally regulated cancers, such as prostate cancer and breast cancer.

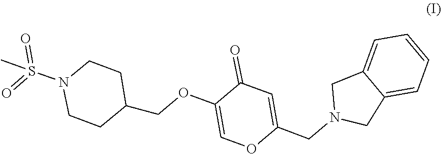 [SMILES: CS(=O)(=O)N1CCC(COc2coc(CN3Cc4ccccc4C3)cc2=O)CC1]

Typically, to enable the efficient development of solid dosage forms, a form of the active ingredient is sought that has a balance of desired properties, such as crystallinity, lack of polymorphism, high melting point, solid-state stability, compressibility and lack of hygroscopicity together with satisfactory solubility. For example, it is desired that a form of the active ingredient, which has the requisite bioavailability, also has sufficient stability such that it does not degrade or convert during manufacture or storage of the pharmaceutical composition to a different form, which has different properties.

Thus, one or more forms of compound (I) are desired having properties and stability that allow a large scale manufacture of marketable pharmaceutical product suitable for the treatment of diseases such as cancer.

### SUMMARY OF THE INVENTION

It has been found that compound (I) can exist in one or more crystalline salt forms that have necessary properties, including stability and processability, that allow their use in large scale manufacture of pharmaceutical products such as tablets or capsules.

In one aspect, the present disclosure provides a salt of compound (I) with p-toluenesulfonic acid, 2-naphthalenesulfonic acid, 1,5-naphthalenedisulfonic acid, hydrobromic acid, nitric acid, benzenesulfonic acid, hydrochloric acid, maleic acid, 1,2-ethanedisulfonic acid, oxalic acid, ethanesulfonic acid, sulfuric acid and methanesulfonic acid.

In another aspect, the present disclosure provides the above salts in crystalline form.

In particular, the present disclosure provides p-toluenesulfonic acid salt, 2-naphthalenesulfonic acid salt, 1,5-naphthalenedisulfonic acid salt and hydrobromic acid salt of compound (I). These salts are crystalline and exhibit particularly high melting point, lack of polymorphism, low weight loss during heating and excellent sold-state stability in pharmaceutical dosage forms such as tablets.

In another aspect, the present disclosure provides a method for the treatment of diseases where CYP11A1 inhibition is desired, particularly in the treatment of hormonally regulated cancers, such as prostate cancer and breast cancer, comprising administering to a subject in need thereof a therapeutically effective amount of any of the above salts of compound (I) or a crystalline form thereof.

In yet another aspect, the present disclosure provides pharmaceutical compositions, particularly in the form of a tablet or a capsule, comprising any of the above salts of compound (I) or a crystalline for thereof together with one or more excipients. In yet another aspect, the present disclosure provides such compositions for use in the treatment of hormonally regulated cancers, such as prostate cancer and breast cancer

### BRIEF DESCRIPTION OF THE DRAWINGS

FIG. 1 shows the X-ray powder diffraction (XRPD) pattern of the crystalline form 1 of p-toluenesulfonic acid salt of compound (I).

FIG. 2 shows the differential scanning calorimetry (DSC) thermogram of the crystalline form 1 of p-toluenesulfonic acid salt of compound (I).

FIG. 3 shows the X-ray powder diffraction (XRPD) pattern of the crystalline form 1 of 2-naphthalenesulfonic acid salt of compound (I).

FIG. 4 shows the differential scanning calorimetry (DSC) thermogram of the crystalline form 1 of 2-naphthalenesulfonic acid salt of compound (I).

FIG. 5 shows the X-ray powder diffraction (XRPD) pattern of the crystalline form 1 of 1,5-naphthalenedisulfonic acid salt of compound (I).

FIG. 6 shows the differential scanning calorimetry (DSC) thermogram of the crystalline form 1 of 1,5-naphthalenedisulfonic acid salt of compound (I).

FIG. 7 shows the X-ray powder diffraction (XRPD) pattern of the crystalline form 1 of hydrobromic acid salt of compound (I).

FIG. 8 shows the differential scanning calorimetry (DSC) thermogram of the crystalline form 1 of hydrobromic acid salt of compound (I).

FIG. 9 shows the X-ray powder diffraction (XRPD) pattern of the crystalline form 1 of nitric acid salt of compound (I).

FIG. 10 shows the differential scanning calorimetry (DSC) thermogram of the crystalline form 1 of nitric acid salt of compound (I).

FIG. 11 shows the X-ray powder diffraction (XRPD) pattern of the crystalline form 1 of benzenesulfonic acid salt of compound (I).

FIG. 12 shows the differential scanning calorimetry (DSC) thermogram of the crystalline form 1 of benzenesulfonic acid salt of compound (I).

FIG. 13 shows the X-ray powder diffraction (XRPD) pattern of the crystalline form 1 of hydrochloric acid salt of compound (I).

FIG. 14 shows the X-ray powder diffraction (XRPD) pattern of the crystalline form 2 of hydrochloric acid salt of compound (I).

FIG. 15 shows the X-ray powder diffraction (XRPD) pattern of the crystalline form 3 of hydrochloric acid salt of compound (I).

FIG. 16 shows the differential scanning calorimetry (DSC) thermogram of the crystalline form 3 of hydrochloric acid salt of compound (I).

FIG. 17 shows the X-ray powder diffraction (XRPD) pattern of the crystalline form 1 of maleic acid salt of compound (I).

FIG. 18 shows the X-ray powder diffraction (XRPD) pattern of the crystalline form 2 of maleic acid salt of compound (I).

FIG. 19 shows the X-ray powder diffraction (XRPD) pattern of the crystalline form 3 of maleic acid salt of compound (I).

FIG. 20 shows the differential scanning calorimetry (DSC) thermogram of the crystalline form 3 of maleic acid salt of compound (I).

FIG. 21 shows the X-ray powder diffraction (XRPD) pattern of the crystalline form 1 of 1,2-ethanedisulfonic acid salt of compound (I).

FIG. 22 shows the differential scanning calorimetry (DSC) thermogram of the crystalline form 1 of 1,2-ethanedisulfonic acid salt of compound (I).

FIG. 23 shows the X-ray powder diffraction (XRPD) pattern of the crystalline form 1 of oxalic acid salt of compound (I).

FIG. 24 shows the differential scanning calorimetry (DSC) thermogram of the crystalline form 1 of oxalic acid salt of compound (I).

FIG. 25 shows the X-ray powder diffraction (XRPD) pattern of the crystalline form 2 of oxalic acid salt of compound (I).

FIG. 26 shows the X-ray powder diffraction (XRPD) pattern of the crystalline form 1 of ethanesulfonic acid salt of compound (I).

FIG. 27 shows the X-ray powder diffraction (XRPD) pattern of the crystalline form 1 of sulfuric acid salt of compound (I).

FIG. 28 shows the X-ray powder diffraction (XRPD) pattern of the crystalline form 2 of sulfuric acid salt of compound (I).

FIG. 29 shows the differential scanning calorimetry (DSC) thermogram of the crystalline form 2 of sulfuric acid salt of compound (I).

FIG. 30 shows the X-ray powder diffraction (XRPD) pattern of the crystalline form 1 of methanesulfonic acid salt of compound (I).

FIG. 31 shows the differential scanning calorimetry (DSC) thermogram of the crystalline form 1 of methanesulfonic acid salt of compound (I).

### DETAILED DESCRIPTION OF THE INVENTION

The term “variable hydrate”, as used herein, refers to a crystalline form that can incorporate various numbers of water molecules without disrupting the crystalline lattice. Thus, such crystalline form can incorporate either stoichiometric or non-stoichiometric amounts of water molecules within its lattice structure.

Salt with p-toluenesulfonic Acid

In one aspect, the present disclosure provides a salt of 2-(isoindolin-2-yl-methyl)-5-((1-(methylsulfonyl)piperidin-4-yl)methoxy)-4H-pyran-4-one (I) with p-toluenesulfonic acid, particularly in crystalline form.

The salt with p-toluenesulfonic acid can be prepared, for example, by dissolving compound (I) and p-toluenesulfonic acid monohydrate, for example in equivalent molar amounts, in a suitable solvent. Suitable solvents include, for example, a mixture of acetonitrile and water, for example in the ratio of about 10:1, or a mixture of of ethanol, water and acetonitrile, wherein the amount of ethanol is suitably about 50-60%, the amount of water about 30-40%, and the amount of acetonitrile about 10 —15%, per volume of the solvent. If desired, activated charcoal and/or amine functionalized silica can be added to the mixture. The mixture can be heated, for example to the temperature of about 50-80° C., suitable to about 70-80° C. If charcoal and/or silica is used, the mixture is thereafter filtered. The obtained solution is stirred and cooled, for example to about 0° C., slowly, for example during about 2-5 hours. The precipitated crystalline salt can then be isolated, for example by filtering, washed, and dried at reduced pressure, for example under vacuum at about 40-60° C., for example for about 10-20 hours.

The salt of compound (I) with p-toluenesulfonic acid appears to precipitate in a single crystalline form, here named as crystalline form 1. No other crystalline forms have been found for p-toluenesulfonic acid salt.

The crystalline form of the salt of compound (I) with p-toluenesulfonic acid has been characterized by X-ray powder diffraction (XRPD) studies.

Accordingly, in one aspect, the present disclosure provides crystalline form 1 of the salt of compound (I) with p-toluenesulfonic acid having a X-ray powder diffraction pattern comprising characteristic peaks at about 4.4, 15.2, 18.4, 19.1, 20.8 and 22.4 degrees 2-theta.

In yet another aspect, the present disclosure provides crystalline form 1 of the salt of compound (I) with p-toluenesulfonic acid having a X-ray powder diffraction pattern comprising characteristic peaks at about 4.4, 8.8, 11.4, 15.2, 16.5, 17.1, 18.4, 19.1, 20.8 and 22.4 degrees 2-theta.

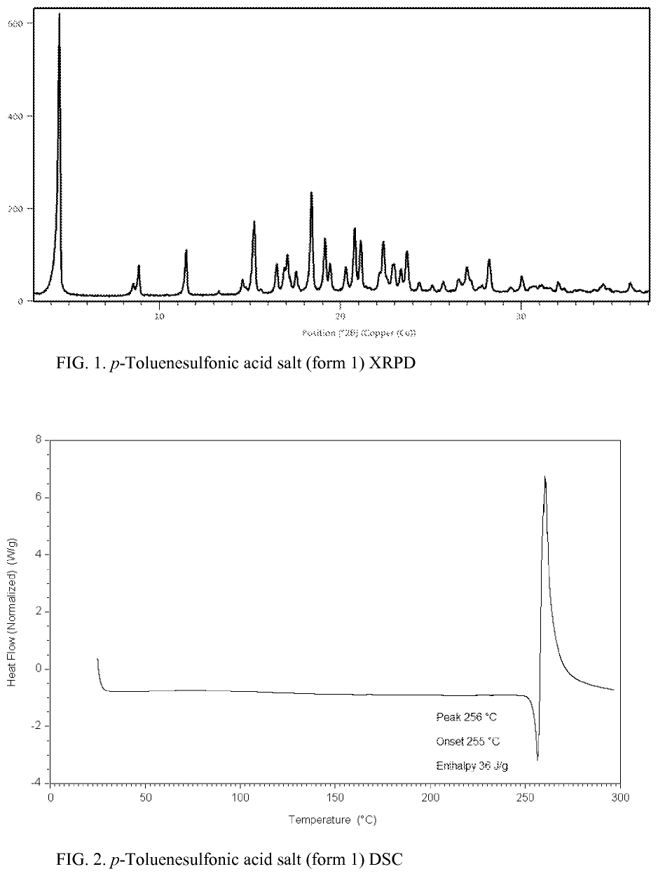

In a further aspect, the crystalline form 1 of the salt of compound (I) with p-toluenesulfonic acid is further characterized by a X-ray powder diffraction pattern as depicted in FIG. 1. In still another aspect, said crystalline form 1 is in the form of a variable hydrate. Thus, there may be small variations in the peak positions shown in FIG. 1 that are related to the variable, non-stoichiometric water content embedded in the crystal structure of the variable hydrate.

Salt with 2-naphthalenesulfonic Acid

In another aspect, the present disclosure provides a salt of 2-(isoindolin-2-yl-methyl)-5-((1-(methylsulfonyl)piperidin-4-yl)methoxy)-4H-pyran-4-one (I) with 2-naphthalenesulfonic acid, particularly in crystalline form.

The salt with 2-naphthalenesulfonic acid can be prepared, for example, by dissolving compound (I) and 2-naphthalenesulfonic acid, for example in equivalent molar amounts, in suitable solvent such as ethanol. If desired, activated charcoal and/or amine functionalized silica can be added to the mixture. The mixture can be heated, for example to the refluxing temperature. If charcoal and/or silica is used, the mixture is thereafter filtered. The obtained solution is stirred and cooled, for example to about room temperature. The precipitated crystalline salt can then be isolated, for example by filtering, washed, and dried at reduced pressure, for example under vacuum at about 40-60° C., for example for about 10-20 hours. If desired, the product can be re-crystallized by dissolving the product, for example, in a mixture of ethanol and water (for example in 30:7 ratio) suitably at refluxing temperature, followed by cooling, for example, to room temperature. The precipitated crystalline salt can then be isolated, for example by filtering, washed, and dried at reduced pressure, for example under vacuum at about 40-60° C., for example for about 10-20 hours.

The salt of compound (I) with 2-naphthalenesulfonic acid appears to precipitate in a single crystalline form, here named as crystalline form 1. No other crystalline forms have been found for 2-naphthalenesulfonic acid salt.

The crystalline form of the salt of compound (I) with 2-naphthalenesulfonic acid has been characterized by X-ray powder diffraction (XRPD) studies.

Accordingly, in one aspect, the present disclosure provides crystalline form 1 of the salt of compound (I) with 2-naphthalenesulfonic acid having a X-ray powder diffraction pattern comprising characteristic peaks at about 4.3, 8.7, 13.0, 18.8 and 27.1 degrees 2-theta.

In yet another aspect, the present disclosure provides crystalline form 1 of the salt of compound (I) with 2-naphthalenesulfonic acid having a X-ray powder diffraction pattern comprising characteristic peaks at about 4.3, 8.7, 13.0, 18.8, 21.7, 27.1 and 35.8 degrees 2-theta.

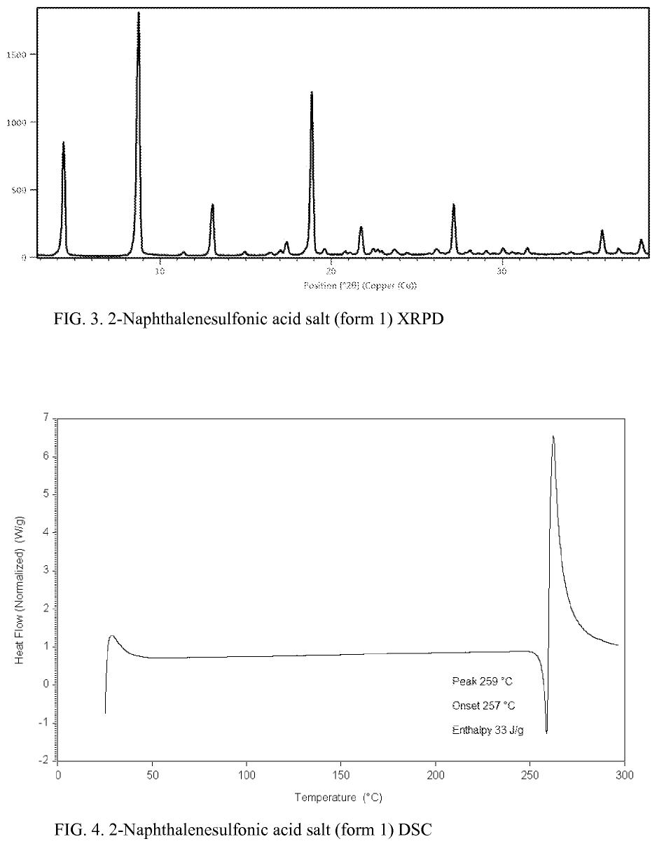

In a further aspect, the crystalline form 1 of the salt of compound (I) with 2-naphthalenesulfonic acid is further characterized by a X-ray powder diffraction pattern as depicted in FIG. 3.

Salt with 1,5-naphthalenedisulfonic Acid

In another aspect, the present disclosure provides a salt of 2-(isoindolin-2-yl-methyl)-5-((1-(methylsulfonyl)piperidin-4-yl)methoxy)-4H-pyran-4-one (I) with 1,5-naphthalenedisulfonic acid, particularly in crystalline form.

The salt with 1,5-naphthalenedisulfonic acid can be prepared, for example, by dissolving compound (I) and 1,5-naphthalenedisulfonic acid, for example in equivalent molar amounts, in suitable solvent. Suitable solvents include, for example, ethanol, a mixture of ethanol and water, or a mixture of acetonitrile and water. If desired, activated charcoal and/or amine functionalized silica can be added to the mixture. The mixture can be heated, for example to the refluxing temperature. If charcoal and/or silica is used, the mixture is thereafter filtered. The obtained solution is stirred and slowly cooled, for example to room temperature. The precipitated crystalline salt can then be isolated, for example by filtering, washed, and dried at reduced pressure, for example under vacuum at about 40-60° C., for example for about 10-20 hours.

The salt of compound (I) with 1,5-naphthalenedisulfonic acid appears to precipitate in a single crystalline form, here named as crystalline form 1. No other crystalline forms have been found for 1,5-naphthalenedisulfonic acid salt.

The crystalline form of the salt of compound (I) with 1,5-naphthalenedisulfonic acid has been characterized by X-ray powder diffraction (XRPD) studies.

Accordingly, in one aspect, the present disclosure provides crystalline form 1 of the salt of compound (I) with 1,5-naphthalenedisulfonic acid having a X-ray powder diffraction pattern comprising characteristic peaks at about 10.6, 17.6, 20.2, 20.4, 22.8 and 24.8 degrees 2-theta.

In yet another aspect, the present disclosure provides crystalline form 1 of the salt of compound (I) with 1,5-naphthalenedisulfonic acid having a X-ray powder diffraction pattern comprising characteristic peaks at about 5.9, 9.2, 10.6, 15.5, 17.1, 17.6, 20.2, 20.4, 22.8 and 24.8 degrees 2-theta.

In a further aspect, the crystalline form 1 of the salt of compound (I) with 1,5-naphthalenedisulfonic acid is further characterized by a X-ray powder diffraction pattern as depicted in FIG. 5.

Salt with Hydrobromic Acid

In another aspect, the present disclosure provides a salt of 2-(isoindolin-2-yl-methyl)-5-((1-(methylsulfonyl)piperidin-4-yl)methoxy)-4H-pyran-4-one (I) with hydrobromic acid, particularly in crystalline form.

The salt with hydrobromic acid can be prepared, for example, by dissolving compound (I) and hydrobromic acid, for example fuming hydrobromic acid in ethanol, in a suitable solvent. Suitable solvents include, for example, ethanol, or a mixture of water with ethanol or isopropanol. If desired, activated charcoal and/or amine functionalized silica can be added to the mixture. The mixture can be heated, for example to the refluxing temperature. If charcoal and/or silica is used, the mixture is thereafter filtered. The obtained solution is stirred and slowly cooled, for example to room temperature. The precipitated crystalline salt can then be isolated, for example by filtering, washed, and dried at reduced pressure, for example under vacuum at about 40-60° C., for example for about 10-20 hours.

The salt of compound (I) with hydrobromic acid appears to precipitate in a single crystalline form, here named as crystalline form 1. No other crystalline forms have been found for hydrobromic acid salt.

The crystalline form of the salt of compound (I) with hydrobromic acid has been characterized by X-ray powder diffraction (XRPD) studies.

Accordingly, in one aspect, the present disclosure provides crystalline form 1 of the salt of compound (I) with hydrobromic acid having a X-ray powder diffraction pattern comprising characteristic peaks at about 5.3, 10.5, 13.6, 18.3, 21.4 and 26.9 degrees 2-theta.

In yet another aspect, the present disclosure provides crystalline form 1 of the salt of compound (I) with hydrobromic acid having a X-ray powder diffraction pattern comprising characteristic peaks at about 5.3, 10.5, 13.6, 16.9, 18.3, 18.8, 21.4, 22.6 and 26.9 degrees 2-theta.

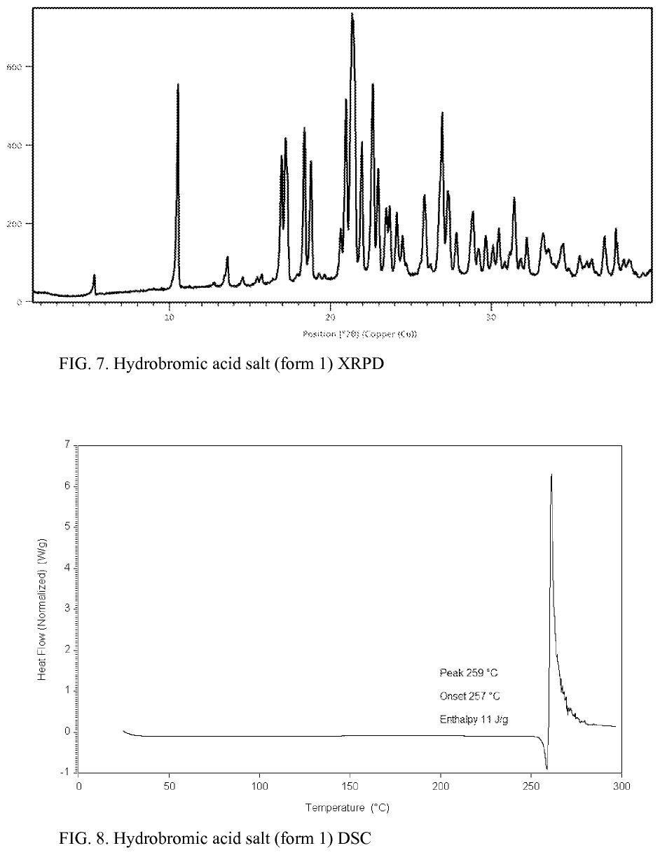

In a further aspect, the crystalline form 1 of the salt of compound (I) with hydrobromic acid is further characterized by a X-ray powder diffraction pattern as depicted in FIG. 7.

Salt with Nitric Acid

In another aspect, the present disclosure provides a salt of 2-(isoindolin-2-yl-methyl)-5-((1-(methylsulfonyl)piperidin-4-yl)methoxy)-4H-pyran-4-one (I) with nitric acid, particularly in crystalline form.

The salt with nitric acid can be prepared, for example, by dissolving compound (I) and nitric acid in a suitable solvent. Suitable solvents include, for example, ethanol, or a mixture of water with ethanol, methanol or isopropanol. If desired, activated charcoal and/or amine functionalized silica can be added to the mixture. The mixture can be heated, for example to the refluxing temperature. If charcoal and/or silica is used, the mixture is thereafter filtered. The obtained solution is stirred and slowly cooled, for example to room temperature. The precipitated crystalline salt can then be isolated, for example by filtering, washed, and dried at reduced pressure, for example under vacuum at about 40-60° C., for example for about 10-20 hours.

The salt of compound (I) with nitric acid appears to precipitate in a single crystalline form, here named as crystalline form 1. No other crystalline forms have been found for nitric acid salt.

The crystalline form of the salt of compound (I) with nitric acid has been characterized by X-ray powder diffraction (XRPD) studies.

Accordingly, in one aspect, the present disclosure provides crystalline form 1 of the salt of compound (I) with nitric acid having a X-ray powder diffraction pattern comprising characteristic peaks at about 10.7, 17.3, 17.9, 20.3, 20.8 and 22.1 degrees 2-theta.

In yet another aspect, the present disclosure provides crystalline form 1 of the salt of compound (I) with nitric acid having a X-ray powder diffraction pattern comprising characteristic peaks at about 10.7, 17.3, 17.6, 17.9, 18.4, 20.3, 20.8, 21.4, 22.1 and 22.7 degrees 2-theta.

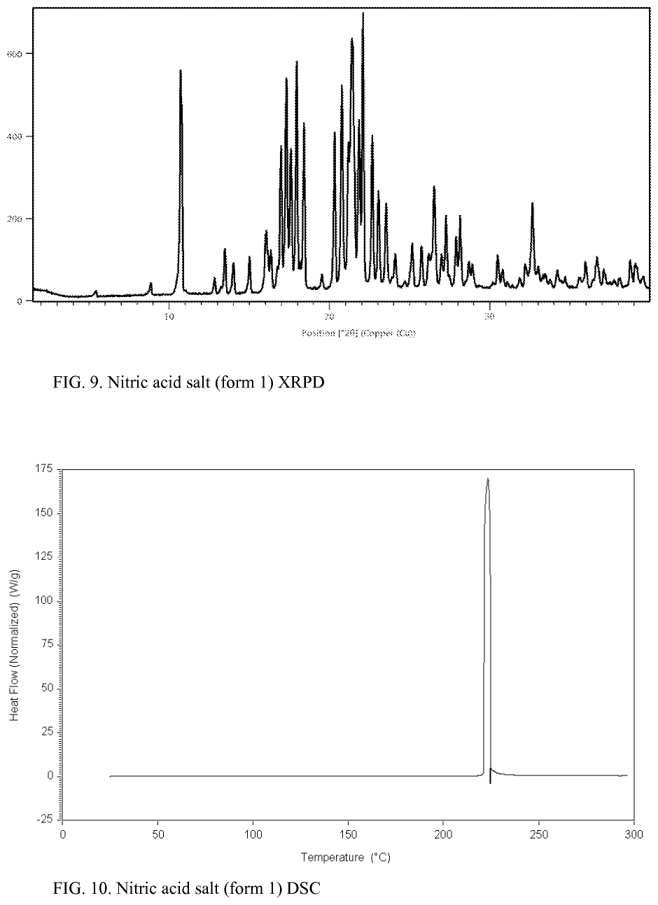

In a further aspect, the crystalline form 1 of the salt of compound (I) with nitric acid is further characterized by a X-ray powder diffraction pattern as depicted in FIG. 9.

Salt with Benzenesulfonic Acid

In another aspect, the present disclosure provides a salt of 2-(isoindolin-2-yl-methyl)-5-((1-(methylsulfonyl)piperidin-4-yl)methoxy)-4H-pyran-4-one (I) with benzenesulfonic acid, particularly in crystalline form.

The salt with benzenesulfonic acid can be prepared, for example, by dissolving compound (I) and benzenesulfonic acid, in a suitable solvent. Suitable solvents include, for example, 2-propanol or a mixture of ethanol and water. If desired, activated charcoal and/or amine functionalized silica can be added to the mixture. The mixture can be heated, for example to the refluxing temperature. If charcoal and/or silica is used, the mixture is thereafter filtered. The obtained solution is stirred and slowly cooled, for example to room temperature. The precipitated crystalline salt can then be isolated, for example by filtering, washed, and dried at reduced pressure, for example under vacuum at about 40-60° C., for example for about 10-20 hours.

The salt of compound (I) with benzenesulfonic acid appears to precipitate in a single crystalline form, here named as crystalline form 1. No other crystalline forms have been found for benzenesulfonic acid salt.

The crystalline form of the salt of compound (I) with benzenesulfonic acid has been characterized by X-ray powder diffraction (XRPD) studies.

Accordingly, in one aspect, the present disclosure provides crystalline form 1 of the salt of compound (I) with benzenesulfonic acid having a X-ray powder diffraction pattern comprising characteristic peaks at about 4.6, 9.1, 13.7 and 19.7 degrees 2-theta.

In yet another aspect, the present disclosure provides crystalline form 1 of the salt of compound (I) with benzenesulfonic acid having a X-ray powder diffraction pattern comprising characteristic peaks at about 4.6, 9.1, 13.7, 15.5, 19.7, 22.9, 24.0 and 27.5 degrees 2-theta.

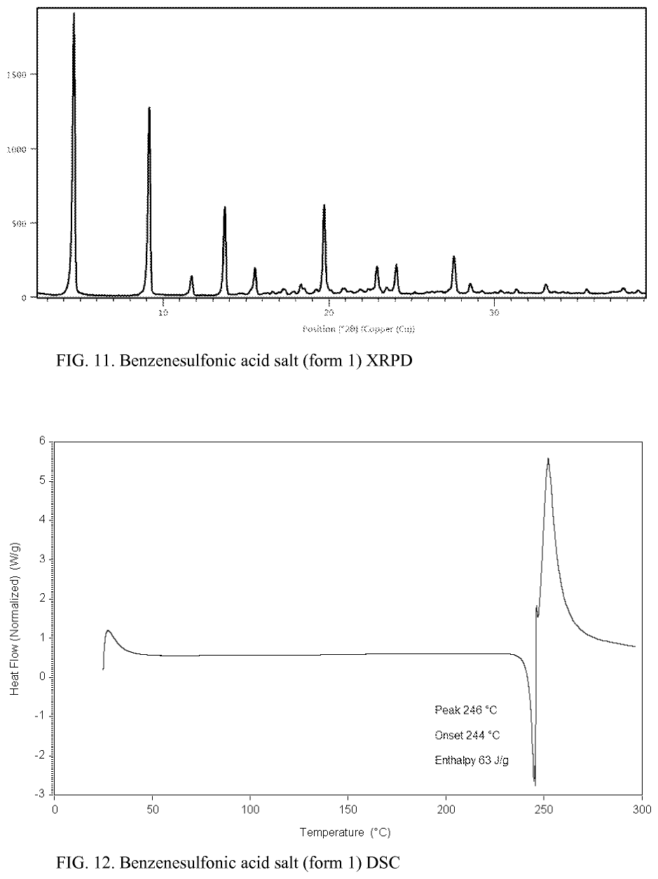

In a further aspect, the crystalline form 1 of the salt of compound (I) with benzenesulfonic acid is further characterized by a X-ray powder diffraction pattern as depicted in FIG. 11.

Salt with Hydrochloric Acid

In another aspect, the present disclosure provides a salt of 2-(isoindolin-2-yl-methyl)-5-((1-(methylsulfonyl)piperidin-4-yl)methoxy)-4H-pyran-4-one (I) with hydrochloric acid, particularly in crystalline form.

The salt with hydrochloric acid has been found to exist in three crystalline forms, named here as crystalline forms 1, 2 and 3. These crystalline forms have been characterized by X-ray powder diffraction (XRPD) studies.

Hydrochloric acid salt crystalline form 1 can be prepared, for example, by dissolving compound (I) and hydrochloric acid, for example hydrochloric acid in diethyl ether, in 2-propanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and slowly cooled, for example to room temperature. The precipitated crystalline salt can then be isolated, for example by filtering, washed, and dried at reduced pressure, for example under vacuum at about 40-60° C., for example for about 10-20 hours, to yield hydrochloric acid salt crystalline form 1.

Thus, in one aspect, the present disclosure provides crystalline form 1 of the salt of compound (I) with hydrochloric acid having a X-ray powder diffraction pattern comprising characteristic peaks at about 4.7, 9.3, 13.9, 16.0, 16.6 and 17.8 degrees 2-theta.

In yet another aspect, the present disclosure provides crystalline form 1 of the salt of compound (I) with hydrochloric acid having a X-ray powder diffraction pattern comprising characteristic peaks at about 4.7, 9.3, 13.9, 16.0, 16.6, 17.8, 21.1, 22.2, 23.3, 24.7 and 26.8 degrees 2-theta.

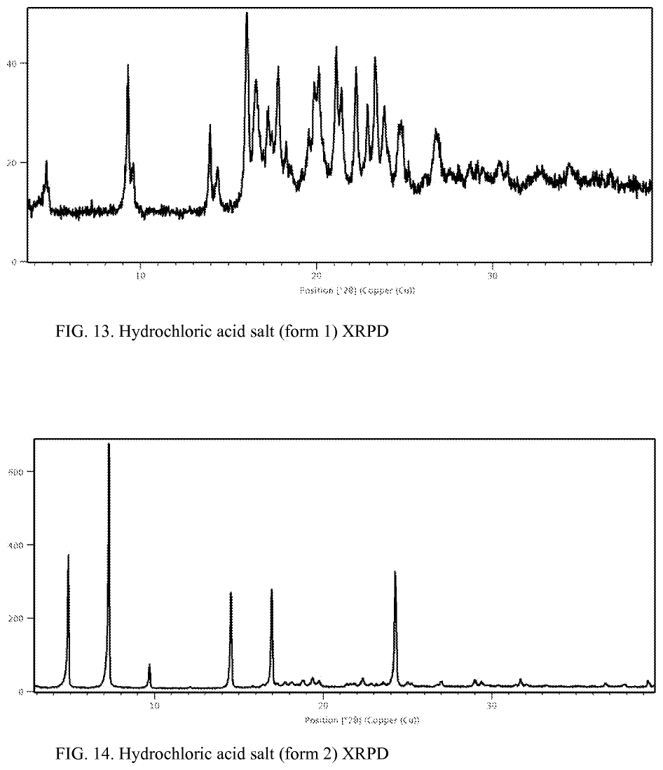

In a further aspect, the crystalline form 1 of the salt of compound (I) with hydrochloric acid is further characterized by a X-ray powder diffraction pattern as depicted in FIG. 13.

Hydrochloric acid salt crystalline form 2 can be prepared, for example, by first dissolving compound (I) and hydrochloric acid, for example hydrochloric acid in cyclopentylmethylether or diethyl ether, in a suitable solvent such as ethanol or 2-propanol. If desired, activated charcoal and/or amine functionalized silica can be added to the mixture. The mixture is suitably heated, for example, to the refluxing temperature. If charcoal and/or silica is used, the mixture is thereafter filtered. The obtained solution is stirred and cooled, for example, to room temperature. The precipitated salt can then be isolated, for example by filtering, washed, and dried at reduced pressure, for example under vacuum at about 40-60° C., for example for about 10-20 hours. The product is then recrystallized by dissolving in a suitable solvent, for example a mixture of 2-propanol and water, for example in the ratio of about 5:4. The mixture is suitably heated, for example, to the refluxing temperature until solids have been dissolved. The obtained solution is stirred and cooled, for example in ice bath. The precipitated crystalline salt can then be isolated, for example by filtering, washed, and dried at reduced pressure, for example under vacuum at about 40-60° C., for example for about 10-20 hours, to yield hydrochloric acid salt crystalline form 2.

Thus, in one aspect, the present disclosure provides crystalline form 2 of the salt of compound (I) with hydrochloric acid having a X-ray powder diffraction pattern comprising characteristic peaks at about 4.9, 7.3, 9.7 and 14.5 degrees 2-theta.

In yet another aspect, the present disclosure provides crystalline form 2 of the salt of compound (I) with hydrochloric acid having a X-ray powder diffraction pattern comprising characteristic peaks at about 4.9, 7.3, 9.7, 14.5, 16.9 and 24.3 degrees 2-theta.

In a further aspect, the crystalline form 2 of the salt of compound (I) with hydrochloric acid is further characterized by a X-ray powder diffraction pattern as depicted in FIG. 14.

Hydrochloric acid salt crystalline form 3 can be prepared, for example, by first dissolving compound (I) and hydrochloric acid, for example hydrochloric acid in water, in suitable solvent such as ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and allowed to cool, for example to room temperature. The precipitated salt can then be isolated, for example by filtering, washed, and dried at reduced pressure, for example under vacuum at about 40-60° C., for example for about 10-20 hours. The product is then recrystallized by dissolving in a suitable solvent, for example a mixture of ethanol and water, for example in the ratio of about 5:1. The mixture is suitably heated, for example, to the refluxing temperature until solids have been dissolved. The obtained solution is stirred and cooled, for example to room temperature. The precipitated crystalline salt can then be isolated, for example by filtering and dried at reduced pressure, for example under vacuum at about 40-60° C., for example for about 10 -20 hours, to yield hydrochloric acid salt crystalline form 3.

Thus, in one aspect, the present disclosure provides crystalline form 3 of the salt of compound (I) with hydrochloric acid having a X-ray powder diffraction pattern comprising characteristic peaks at about 15.8, 19.1, 21.0, 22.5, 29.8 and 32.5 degrees 2-theta.

In yet another aspect, the present disclosure provides crystalline form 3 of the salt of compound (I) with hydrochloric acid having a X-ray powder diffraction pattern comprising characteristic peaks at about 15.8, 19.1, 20.2, 21.0, 22.5, 23.9, 29.8, 32.5 and 34.1 degrees 2-theta.

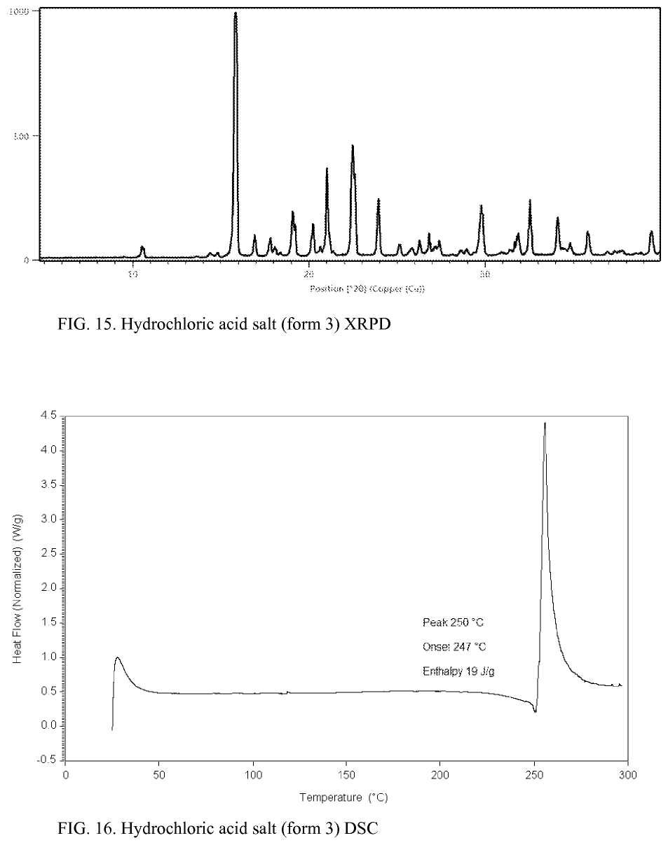

In a further aspect, the crystalline form 3 of the salt of compound (I) with hydrochloric acid is further characterized by a X-ray powder diffraction pattern as depicted in FIG. 15.

Salt with Maleic Acid

In another aspect, the present disclosure provides a salt of 2-(isoindolin-2-ylmethyl)-5-((1-(methylsulfonyl)piperidin-4-yl)methoxy)-4H-pyran-4-one (I) with maleic acid, particularly in crystalline form.

The salt with maleic acid has been found to exist in three crystalline forms, named here as crystalline forms 1, 2 and 3. These crystalline forms have been characterized by X-ray powder diffraction (XRPD) studies.

Maleic acid salt crystalline form 1 can be prepared, for example, by dissolving compound (I) and maleic acid in 2-propanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and slowly cooled, for example, to room temperature. The precipitated crystalline salt can then be isolated, for example by filtering, washed, and dried at reduced pressure, for example under vacuum at about 40-60° C., for example for about 10-20 hours, to yield maleic acid salt crystalline form 1.

Thus, in one aspect, the present disclosure provides crystalline form 1 of the salt of compound (I) with maleic acid having a X-ray powder diffraction pattern comprising characteristic peaks at about 5.5, 10.9, 13.7, 16.4, 21.3 and 21.9 degrees 2-theta.

In yet another aspect, the present disclosure provides crystalline form 1 of the salt of compound (I) with maleic acid having a X-ray powder diffraction pattern comprising characteristic peaks at about 5.5, 10.9, 11.2, 13.7, 16.4, 17.7, 18.8, 19.6, 21.3 and 21.9 degrees 2-theta.

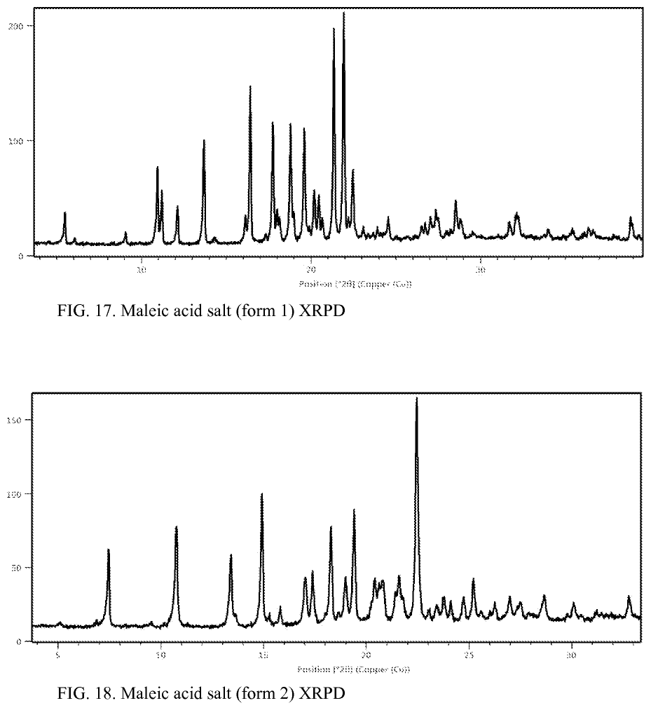

In a further aspect, the crystalline form 1 of the salt of compound (I) with maleic acid is further characterized by a X-ray powder diffraction pattern as depicted in FIG. 17.

Maleic acid salt crystalline form 2 can be prepared, for example, by dissolving compound (I) and maleic acid in ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and slowly cooled, for example, to room temperature. The precipitated crystalline salt can then be isolated, for example by filtering, washed, and dried at reduced pressure, for example under vacuum at about 40-60° C., for example for about 10-20 hours, to yield maleic acid salt crystalline form 2.

Thus, in one aspect, the present disclosure provides crystalline form 2 of the salt of compound (I) with maleic acid having a X-ray powder diffraction pattern comprising characteristic peaks at about 7.4, 10.7, 13.4, 14.9 and 22.4 degrees 2-theta.

In yet another aspect, the present disclosure provides crystalline form 2 of the salt of compound (I) with maleic acid having a X-ray powder diffraction pattern comprising characteristic peaks at about 7.4, 10.7, 13.4, 14.9, 18.3, 19.4 and 22.4 degrees 2-theta.

In a further aspect, the crystalline form 2 of the salt of compound (I) with maleic acid is further characterized by a X-ray powder diffraction pattern as depicted in FIG. 18.

Maleic acid salt crystalline form 3 can be prepared, for example, by first dissolving compound (I) and maleic acid in ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and allowed to cool, for example, to room temperature. The precipitated salt can then be isolated, for example by filtering, washed, and dried at reduced pressure, for example under vacuum at about 40-60° C., for example for about 10-20 hours. The product is then recrystallized by dissolving in a suitable solvent, for example a mixture of ethanol and water, for example in the ratio of about 33:5. The mixture is suitably heated, for example, to the refluxing temperature until solids have been dissolved. The obtained solution is stirred and allowed to cool, for example to room temperature. The precipitated crystalline salt can then be isolated, for example by filtering and dried at reduced pressure, for example under vacuum at about 40-60° C., for example for about 10-20 hours, to yield maleic acid salt crystalline form 3.

Thus, in one aspect, the present disclosure provides crystalline form 3 of the salt of compound (I) with maleic acid having a X-ray powder diffraction pattern comprising characteristic peaks at about 5.9, 11.4, 14.5, 16.2, 23.0 and 23.7 degrees 2-theta.

In yet another aspect, the present disclosure provides crystalline form 3 of the salt of compound (I) with maleic acid having a X-ray powder diffraction pattern comprising characteristic peaks at about 5.9, 11.4, 11.8, 14.5, 16.2, 17.7, 22.7, 23.0, 23.7 and 28.5 degrees 2-theta.

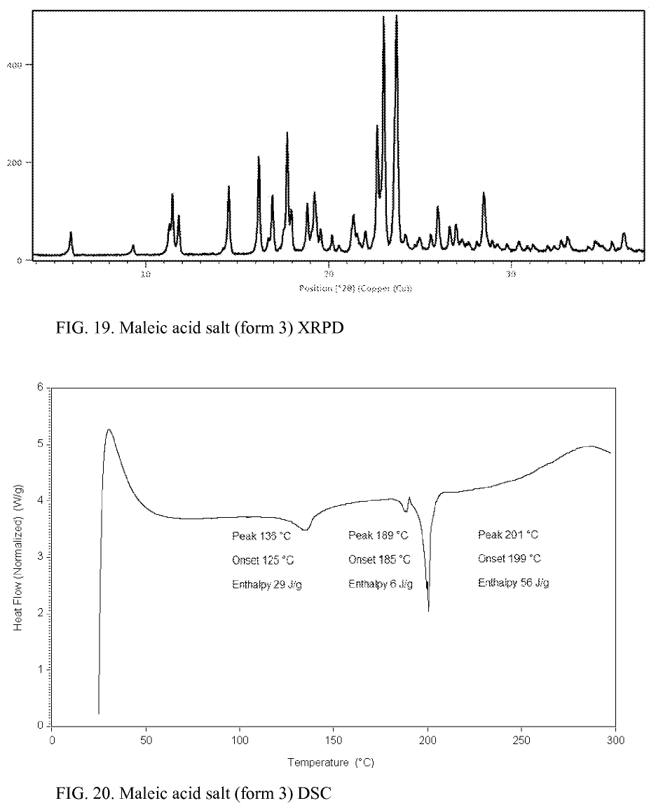

In a further aspect, the crystalline form 3 of the salt of compound (I) with maleic acid is further characterized by a X-ray powder diffraction pattern as depicted in FIG. 19.

Salt with 1,2-ethanedisulfonic Acid

In another aspect, the present disclosure provides a salt of 2-(isoindolin-2-yl-methyl)-5-((1-(methylsulfonyl)piperidin-4-yl)methoxy)-4H-pyran-4-one (I) with 1,2-ethanedisulfonic acid, particularly in crystalline form.

The salt with 1,2-ethanedisulfonic acid can be prepared, for example, by dissolving compound (I) and 1,2-ethanedisulfonic acid, in a suitable solvent. Suitable solvents include, for example, ethanol or a mixture water with ethanol, methanol, isopropanol or acetonitrile. If desired, activated charcoal and/or amine functionalized silica can be added to the mixture. The mixture can be heated, for example to the refluxing temperature. If charcoal and/or silica is used, the mixture is thereafter filtered. The obtained solution is stirred and cooled, for example to room temperature. The precipitated crystalline salt can then be isolated, for example by filtering, washed, and dried at reduced pressure, for example under vacuum at about 40-60° C., for example for about 10-20 hours.

The salt of compound (I) with 1,2-ethanedisulfonic acid appears to precipitate in a single crystalline form, here named as crystalline form 1. No other crystalline forms have been found for 1,2-ethanedisulfonic acid salt.

Thus, in one aspect, the present disclosure provides crystalline form 1 of the salt of compound (I) with 1,2-ethanedisulfonic acid having a X-ray powder diffraction pattern comprising characteristic peaks at about 7.9, 9.1, 10.7, 14.9, 16.8 and 23.7 degrees 2-theta.

In yet another aspect, the present disclosure provides crystalline form 1 of the salt of compound (I) with 1,2-ethanedisulfonic acid having a X-ray powder diffraction pattern comprising characteristic peaks at about 7.9, 9.1, 10.7, 14.9, 15.2, 16.8, 20.5 and 23.7 degrees 2-theta.

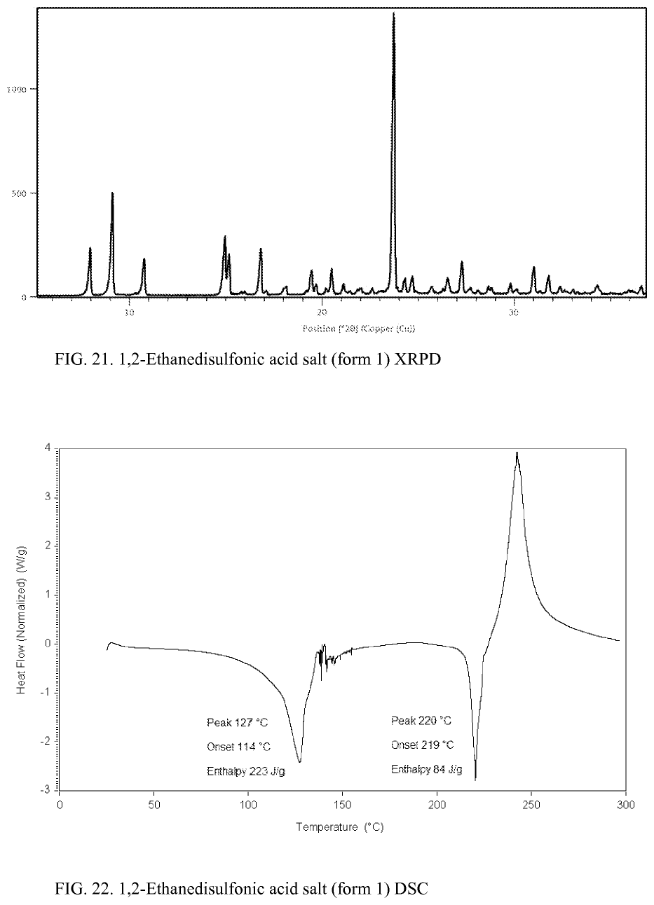

In a further aspect, the crystalline form 1 of the salt of compound (I) with 1,2-ethanedisulfonic acid is further characterized by a X-ray powder diffraction pattern as depicted in FIG. 21.

Salt with Oxalic Acid

In another aspect, the present disclosure provides a salt of 2-(isoindolin-2-ylmethyl)-5-((1-(methylsulfonyl)piperidin-4-yl)methoxy)-4H-pyran-4-one (I) with oxalic acid, particularly in crystalline form.

The salt with oxalic acid has been found to exist in two crystalline forms, named here as crystalline form 1 and 2. These crystalline forms have been characterized by X-ray powder diffraction (XRPD) studies.

Oxalic acid salt crystalline form 1 can be prepared, for example, by dissolving compound (I) and oxalic acid in ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and slowly cooled, for example, to room temperature. The precipitated crystalline salt can then be isolated, for example by filtering, washed, and dried at reduced pressure, for example under vacuum at about 40-60° C., for example for about 10-20 hours, to yield oxalic acid salt crystalline form 1.

Thus, in one aspect, the present disclosure provides crystalline form 1 of the salt of compound (I) with oxalic acid having a X-ray powder diffraction pattern comprising characteristic peaks at about 6.1, 11.7, 17.0, 18.7, 19.3 and 25.2 degrees 2-theta.

In yet another aspect, the present disclosure provides crystalline form 1 of the salt of compound (I) with oxalic acid having a X-ray powder diffraction pattern comprising characteristic peaks at about 6.1, 6.6, 11.7, 13.2, 17.0, 18.7, 19.3, 22.2 and 25.2 degrees 2-theta.

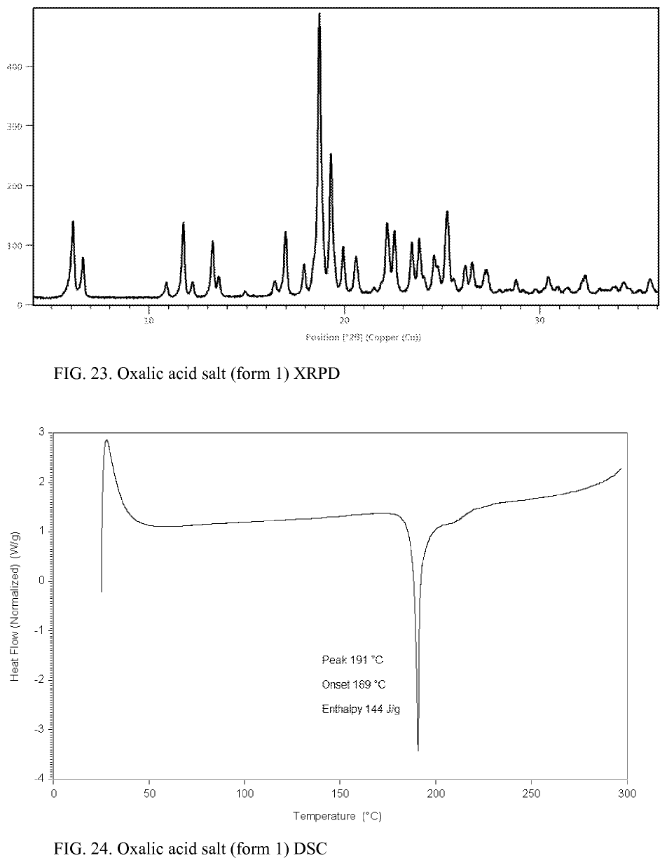

In a further aspect, the crystalline form 1 of the salt of compound (I) with oxalic acid is further characterized by a X-ray powder diffraction pattern as depicted in FIG. 23.

Oxalic acid salt crystalline form 2 can be prepared, for example, by dissolving compound (I) and oxalic acid in acetonitrile. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and slowly cooled, for example, to room temperature. The precipitated crystalline salt can then be isolated, for example by filtering, washed, and dried at reduced pressure, for example under vacuum at about 40-60° C., for example for about 10-20 hours, to yield oxalic acid salt crystalline form 2.

Thus, in one aspect, the present disclosure provides crystalline form 2 of the salt of compound (I) with oxalic acid having a X-ray powder diffraction pattern comprising characteristic peaks at about 4.2, 5.4, 6.4, 11.1 and 15.3 degrees 2-theta.

In yet another aspect, the present disclosure provides crystalline form 2 of the salt of compound (I) with oxalic acid having a X-ray powder diffraction pattern comprising characteristic peaks at about 4.2, 5.4, 6.4, 11.1, 15.3, 19.5, 18.9, 20.2 and 22.2 degrees 2-theta.

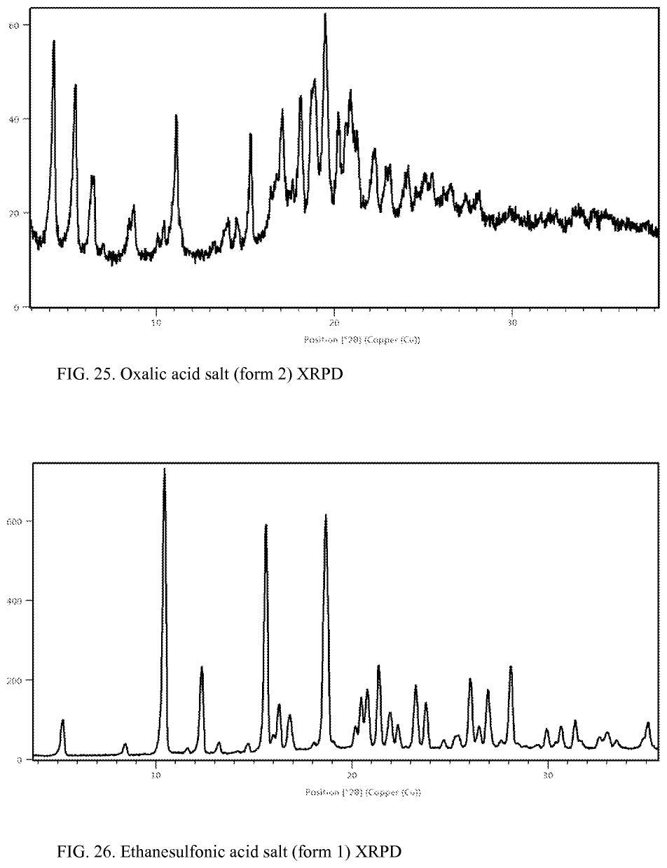

In a further aspect, the crystalline form 2 of the salt of compound (I) with oxalic acid is further characterized by a X-ray powder diffraction pattern as depicted in FIG. 25.

Salt with Ethanesulfonic Acid

In another aspect, the present disclosure provides a salt of 2-(isoindolin-2-ylmethyl)-5-((1-(methylsulfonyl)piperidin-4-yl)methoxy)-4H-pyran-4-one (I) with ethanesulfonic acid, particularly in crystalline form.

Ethanesulfonic acid salt crystalline form 1 can be prepared, for example, by dissolving compound (I) and ethanesulfonic acid in ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and cooled, for example, to room temperature. The precipitated crystalline salt can then be isolated, for example by filtering, washed, and dried at reduced pressure, for example under vacuum at about 40-60° C., for example for about 10-20 hours, to yield ethanesulfonic acid salt crystalline form 1.

The salt of compound (I) with ethanesulfonic acid appears to precipitate in a single crystalline form, here named as crystalline form 1. No other crystalline forms have been found for ethanesulfonic acid salt.

Thus, in one aspect, the present disclosure provides crystalline form 1 of the salt of compound (I) with ethanesulfonic acid having a X-ray powder diffraction pattern comprising characteristic peaks at about 10.4, 12.3, 15.6, 18.7 and 28.1 degrees 2-theta.

In yet another aspect, the present disclosure provides crystalline form 1 of the salt of compound (I) with ethanesulfonic acid having a X-ray powder diffraction pattern comprising characteristic peaks at about 5.2, 10.4, 12.3, 15.6, 18.7, 21.4, 26.0, 26.9 and 28.1 degrees 2-theta.

In a further aspect, the crystalline form 1 of the salt of compound (I) with ethanesulfonic acid is further characterized by a X-ray powder diffraction pattern as depicted in FIG. 26.

Salt with Sulphuric Acid

In another aspect, the present disclosure provides a salt of 2-(isoindolin-2-ylmethyl)-5-((1-(methylsulfonyl)piperidin-4-yl)methoxy)-4H-pyran-4-one (I) with sulfuric acid, particularly in crystalline form.

The salt with sulfuric acid has been found to exist in two crystalline forms, named here as crystalline form 1 and 2. These crystalline forms have been characterized by X-ray powder diffraction (XRPD) studies.

Sulfuric acid salt crystalline form 1 can be prepared, for example, by dissolving compound (I) and sulfuric acid, for example sulphuric acid in ethanol, in a mixture of acetonitrile or a mixture of acetonitrile and water, for example in ratio 25:2. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and cooled, for example, to room temperature. The precipitated crystalline salt can then be isolated, for example by filtering, washed, and dried at reduced pressure, for example under vacuum at about 40-60° C., for example for about 10-20 hours, to yield sulfuric acid salt crystalline form 1.

Thus, in one aspect, the present disclosure provides crystalline form 1 of the salt of compound (I) with sulfuric acid having a X-ray powder diffraction pattern comprising characteristic peaks at about 5.5, 11.1, 17.5, 20.8 and 22.1 degrees 2-theta.

In yet another aspect, the present disclosure provides crystalline form 1 of the salt of compound (I) with sulfuric acid having a X-ray powder diffraction pattern comprising characteristic peaks at about 5.5, 11.1, 17.5, 17.8, 20.8, 21.3, 22.1 and 23.4 degrees 2-theta.

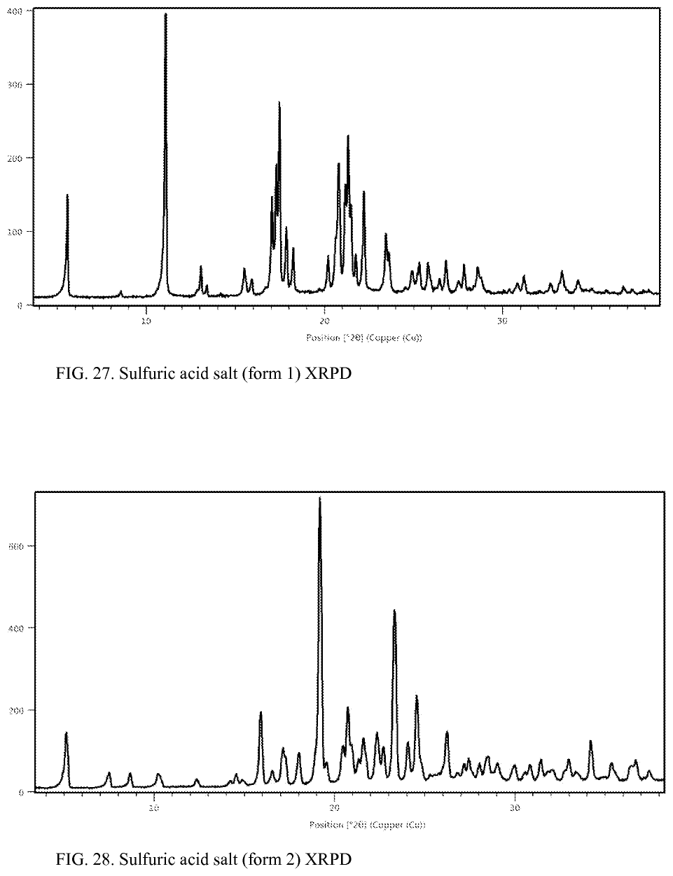

In a further aspect, the crystalline form 1 of the salt of compound (I) with sulfuric acid is further characterized by a X-ray powder diffraction pattern as depicted in FIG. 27.

Sulfuric acid salt crystalline form 2 can be prepared, for example, by dissolving compound (I) and sulfuric acid, for example sulphuric acid in ethanol, in ethanol. If desired, activated charcoal and/or amine functionalized silica can be added to the mixture. The mixture is suitably heated, for example to the refluxing temperature. If charcoal and/or silica is used, the mixture is thereafter filtered. The obtained solution is stirred and cooled, for example, to room temperature. The precipitated crystalline salt can then be isolated, for example by filtering, washed, and dried at reduced pressure, for example under vacuum at about 40-60° C., for example for about 10-20 hours, to yield sulfuric acid salt crystalline form 2.

Thus, in one aspect, the present disclosure provides crystalline form 2 of the salt of compound (I) with sulfuric acid having a X-ray powder diffraction pattern comprising characteristic peaks at about 5.1, 15.9, 19.1, 20.7 and 23.3 degrees 2-theta.

In yet another aspect, the present disclosure provides crystalline form 2 of the salt of compound (I) with sulfuric acid having a X-ray powder diffraction pattern comprising characteristic peaks at about 5.1, 15.9, 19.1, 20.7, 23.3, 24.5 and 26.2 degrees 2-theta.

In a further aspect, the crystalline form 2 of the salt of compound (I) with sulfuric acid is further characterized by a X-ray powder diffraction pattern as depicted in FIG. 28.

Salt with Methanesulfonic Acid

In another aspect, the present disclosure provides a salt of 2-(isoindolin-2-ylmethyl)-5-((1-(methylsulfonyl)piperidin-4-yl)methoxy)-4H-pyran-4-one (I) with methanesulfonic acid, particularly in crystalline form.

Methanesulfonic acid salt crystalline form 1 can be prepared, for example, by first dissolving compound (I) and methanesulfonic acid, for example methanesulfonic acid in ethanol, in suitable solvent such as ethanol. If desired, activated charcoal and/or amine functionalized silica can be added to the mixture. The mixture is suitably heated, for example to the refluxing temperature. If charcoal and/or silica is used, the mixture is thereafter filtered. The solvent of the obtained solution can be removed in vacuo. The remaining product can then be recrystallized by dissolving in a suitable solvent, for example a mixture of acetonitrile and water, for example in the ratio of about 125:6. The mixture is suitably heated, for example, to the refluxing temperature until solids have been dissolved. The obtained solution is stirred and cooled, for example to room temperature. The precipitated crystalline salt can then be isolated, for example by filtering and dried at reduced pressure, for example under vacuum at about 40-60° C., for example for about 10-20 hours, to yield methanesulfonic acid salt crystalline form 1.

The salt of compound (I) with methanesulfonic acid appears to precipitate in a single crystalline form, here named as crystalline form 1. No other crystalline forms have been found for methanesulfonic acid salt.

Thus, in one aspect, the present disclosure provides crystalline form 1 of the salt of compound (I) with methanesulfonic acid having a X-ray powder diffraction pattern comprising characteristic peaks at about 5.2, 10.3, 19.3 and 24.2 degrees 2-theta.

In yet another aspect, the present disclosure provides crystalline form 1 of the salt of compound (I) with methanesulfonic acid having a X-ray powder diffraction pattern comprising characteristic peaks at about 5.2, 10.3, 17.2, 18.1, 19.3 and 24.2 degrees 2-theta.

In a further aspect, the crystalline form 1 of the salt of compound (I) with methanesulfonic acid is further characterized by a X-ray powder diffraction pattern as depicted in FIG. 30.

The above XRPD peak positions refer to values, when measured using CuKα radiation (λ=1.5418 Å). It is recognized by the skilled person that the X-ray powder diffraction pattern peak positions referred to herein can be subject to variations of ±0.2 degrees 2-theta according to various factors such as temperature, sample handling and instrumentation used.

The above crystalline salts of compound (I) can be formulated into pharmaceutical dosage forms such as tablets, capsules, granules, powders or suspensions together with excipients which are known in the art.

Thus, in one aspect, the present disclosure provides a pharmaceutical composition comprising any of the above salts of compound (I) or crystalline forms thereof together with one or more excipients, particularly in the form of a tablet or a capsule.

In another aspect, the present disclosure provides substantially pure crystalline forms of the salts of compound (I), as disclosed above, wherein at least 90%, preferably at least 95%, more preferably at least 98%, per weight of the salt of compound (I) is present in said crystalline form.

The invention is further illustrated by the following non-limiting examples.

### Analytical Methods

XRPD measurements were performed with the X-ray powder diffractometer PANalytical X'Pert PRO at room temperature using copper filled X-ray tube (45 kV×40 mA) as the X-ray source, CuKα (λ=1.5418 Å), a fixed 1° anti-scatter slit, a programmable divergence slit with 10 mm irradiated length, and the real time multiple strip detector X'Celerator. Data collection was done in 0.017° steps at a scan speed of 0.1°/s in the range of 3-40° 2θ.

Differential scanning calorimetry (DSC) was carried out on a TA Instruments Discovery DSC, under nitrogen flow (50 ml/min), at 10° C./min constant heating rate, in a high pressure sample pan.

Melting point was determined by observing the phase change during hot stage microscopy in open chamber with heating rate of 10° C./min.

Weight loss of the salts during heating was determined by thermogravimetric analysis (TGA) thermograms collected on TGA equipment (TA Instruments). Heating rate 10° C./min, 25-300° C., open pan.

Single-crystal diffraction data were collected on a Rigaku Oxford Diffraction SuperNova dual-wavelength diffractometer with the operating mirror monochromated Cu Kα (λ=1.5418 Å) or Mo Kα radiation mode (λ=0.7107 Å). X-ray data collection was monitored, and all data were corrected for Lorentzian, polarization, and absorption effects using the CrysAlisPro program. The Olex2 program was used for the crystal structure solution and refinement, SHELXS97 for structure solution, and SHELXL for full-matrix least-squares refinement on F².

### EXAMPLE 1

### p-Toluenesulfonic Acid Salt Crystalline Form 1

To a reactor under nitrogen was added ethanol (117 ml), water (74 ml), acetonitrile (25 ml), 2-(isoindolin-2-ylmethyl)-5-((1-(methylsulfonyl)piperidin-4-yl)-methoxy)-4H-pyran-4-one (I) (20 g), p-toluenesulfonic acid monohydrate (9.5 g), activated charcoal (Norit SX ultra, 2 g) and amine functionalized silica (SiliaMetS Triamine, 1 g). The mixture was heated to about 75° C. and agitated for 1 h. The mixture was filtered through a depth filter in to a clean reactor. The filter cake was washed with preheated mixture of 1:1 MeCN/water (10 ml). The temperature of the filtrate was adjusted to 55±5° C. and seed crystals were added. The mixture was allowed to stir for about 30 minutes and the cooled to 0±5° C. over 3 hours. The mass was stirred for 1 hour prior to filtration. The product was washed with absolute ethanol (60 ml) and dried under vacuum at 40-60° C. to yield 23.9 g (84.7%) of pure white plate-like crystals of p-toluenesulfonic acid salt of compound (I). The product was analysed by XRPD and was found to be crystalline form 1 (Table 1). The X-ray powder diffraction pattern of form 1 is depicted in FIG. 1 and the differential scanning calorimetry (DSC) thermogram in FIG. 2. 1H NMR (400 MHz, DMSO-d6): δ ppm 1.21-1.38 (m, 2 H), 1.78-1.91 (m, 3 H), 2.28 (s, 3 H), 2.66-2.77 (m, 2 H), 2.86 (s, 3 H), 3.54-3.64 (m, 2 H), 3.71-3.77 (m, 2 H), 4.60-4.80 (m, 4 H), 6.65 (s, 1 H), 7.04-7.13 (m, 2 H), 7.34-7.43 (m, 4 H), 7.44-7.49 (m, 2 H), 8.24 (s, 1 H), 10.90-11.59 (m, 1 H).

TABLE 1
X-ray powder reflections (up to 40° 2θ) and intensities (normalized)
of p-toluenesulfonic acid salt crystalline form 1. The value 2θ
[°] represents the diffraction angle in degrees and the value
d [Å] represents the specified distances in Å between the lattice planes.

| 2θ [°] | d [Å] | I/Iₒ [%] |
|---|---|---|
| 4.42 | 19.97 | 100 |
| 8.53 | 10.35 | 4 |
| 8.83 | 10.01 | 11 |
| 11.44 | 7.73 | 17 |
| 13.25 | 6.68 | 1 |
| 14.58 | 6.07 | 6 |
| 15.19 | 5.83 | 28 |
| 15.60 | 5.68 | 1 |
| 16.45 | 5.39 | 12 |
| 16.87 | 5.25 | 8 |
| 17.06 | 5.19 | 15 |
| 17.53 | 5.05 | 9 |
| 18.37 | 4.83 | 41 |
| 19.12 | 4.64 | 22 |
| 19.41 | 4.57 | 11 |
| 20.27 | 4.38 | 10 |
| 20.76 | 4.28 | 26 |
| 21.10 | 4.21 | 21 |
| 22.13 | 4.01 | 3 |
| 22.35 | 3.98 | 20 |
| 22.86 | 3.89 | 9 |
| 22.97 | 3.87 | 8 |
| 23.31 | 3.81 | 9 |
| 23.65 | 3.76 | 17 |
| 24.33 | 3.66 | 4 |
| 25.04 | 3.55 | 3 |
| 25.66 | 3.47 | 4 |
| 26.50 | 3.36 | 5 |
| 26.97 | 3.30 | 10 |
| 27.78 | 3.21 | 2 |
| 28.19 | 3.16 | 14 |
| 29.38 | 3.04 | 2 |
| 30.00 | 2.98 | 6 |
| 30.60 | 2.92 | 1 |
| 31.08 | 2.87 | 2 |
| 32.01 | 2.79 | 4 |
| 33.19 | 2.70 | 1 |
| 34.49 | 2.60 | 3 |
| 35.99 | 2.49 | 4 |

### EXAMPLE 2

### p-Toluenesulfonic Acid Salt Crystalline Form 1 (Alternative Method)

p-Toluenesulfonic acid monohydrate (3.9 mmol, 1 eq.) was added to a solution of 2-(isoindolin-2-ylmethyl)-5-((1-(methylsulfonyl)piperidin-4-yl)methoxy)-4H-pyran-4-one (I) (3.9 mmol, 1 eq.) in 33 ml of acetonitrile:water 10:1. The slurry was stirred at refluxing temperature until all solids were dissolved. The mixture was stirred and allowed to cool to room temperature followed by stirring in an ice-bath. The precipitated solid was filtered, washed twice with acetonitrile, and dried under vacuum at 40° C. for 16 h to yield p-toluenesulfonic acid salt of compound (I) (1.5 g, 66%). The product was analysed by XRPD and was found to be crystalline form 1 (Table 1). 1H NMR (400 MHz, DMSO-d6): δ ppm 1.21-1.38 (m, 2 H), 1.78-1.91 (m, 3 H), 2.28 (s, 3 H), 2.66-2.77 (m, 2 H), 2.86 (s, 3 H), 3.54-3.64 (m, 2 H), 3.71 -3.77 (m, 2 H), 4.60-4.80 (m, 4 H), 6.65 (s, 1 H), 7.04-7.13 (m, 2 H), 7.34-7.43 (m, 4 H), 7.44-7.49 (m, 2 H), 8.24 (s, 1 H), 10.90-11.59 (m, 1 H).

### EXAMPLE 3

### 2-Naphthalenesulfonic Acid Salt Crystalline Form 1

2-Naphthalenesulfonic acid (1.741 g, 5.85 mmol) in 5 ml of ethanol was added to a solution of 2-(isoindolin-2-ylmethyl)-5-((1-(methylsulfonyl)piperidin-4-yl)methoxy)-4H-pyran-4-one (I) (2.45 g, 5.85 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h. The product was then dissolved in 60 ml of ethanol:water (30:7) at refluxing temperature. The mixture was allowed to cool to room temperature. The precipitated solid was isolated by filtering, washed with ethanol:water (30:7) and dried under vacuum at 40° C. for 16 h to yield 2-naphthalenesulfonic acid salt of compound (I) (4.24 g, 116%). The product was analysed by XRPD and was found to be crystalline form 1 (Table 2). The X-ray powder diffraction pattern of form 1 is depicted in FIG. 3 and the differential scanning calorimetry (DSC) thermogram in FIG. 4. 1H NMR (400 MHz, DMSO-d6): δ ppm 1.20-1.38 (m, 2 H), 1.77-1.91 (m, 3 H), 2.73 (td, 2.11 Hz, 3 H), 2.87 (s, 3 H), 3.59 (br d, 2 H), 3.74 (d, 2 H), 4.58-4.86 (m, 4 H), 6.64 (s, 1 H), 7.39 (br d, 4 H), 7.49-7.56 (m, 2 H), 7.70 (dd, 1 H), 7.86 (d, 1 H), 7.88-7.93 (m, 1 H), 7.94 -35 7.99 (m, 1 H), 8.13 (d, 1 H), 8.24 (s, 1 H), 10.86-11.70 (m, 1 H).

TABLE 2
X-ray powder reflections (up to 40° 2θ) and intensities (normalized)
of 2-naphthalenesulfonic acid salt crystalline form 1. The value 2θ
[°] represents the diffraction angle in degrees and the value
d [Å] represents the specified distances in Å between the lattice planes.

| 2θ [°] | d [Å] | I/Iₒ [%] |
|---|---|---|
| 4.32 | 20.45 | 48 |
| 4.40 | 20.07 | 24 |
| 8.62 | 10.25 | 53 |
| 8.74 | 10.11 | 100 |
| 11.36 | 7.79 | 2 |
| 13.03 | 6.79 | 24 |
| 14.93 | 5.93 | 2 |
| 16.41 | 5.40 | 1 |
| 17.00 | 5.21 | 2 |
| 17.37 | 5.10 | 6 |
| 18.84 | 4.71 | 79 |
| 19.59 | 4.53 | 3 |
| 20.79 | 4.27 | 2 |
| 21.09 | 4.21 | 1 |
| 21.72 | 4.09 | 14 |
| 22.42 | 3.96 | 3 |
| 22.70 | 3.91 | 2 |
| 22.94 | 3.87 | 2 |
| 23.68 | 3.75 | 3 |
| 24.41 | 3.64 | 1 |
| 25.81 | 3.45 | 0 |
| 26.15 | 3.41 | 3 |
| 27.14 | 3.28 | 26 |
| 28.07 | 3.18 | 2 |
| 29.03 | 3.07 | 2 |
| 30.02 | 2.97 | 3 |
| 30.55 | 2.92 | 1 |
| 30.91 | 2.89 | 1 |
| 31.44 | 2.84 | 3 |
| 33.49 | 2.67 | 1 |
| 33.98 | 2.64 | 1 |
| 34.95 | 2.56 | 1 |
| 35.82 | 2.51 | 12 |
| 36.78 | 2.44 | 3 |
| 38.10 | 2.36 | 8 |

### EXAMPLE 4

### 1,5-Naphthalenedisulfonic acid salt crystalline form 1

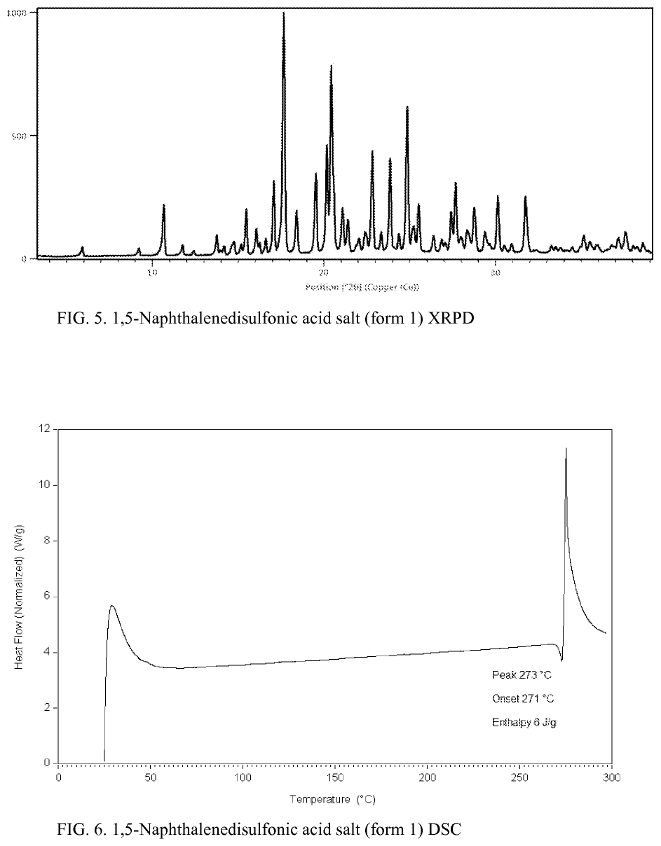

1,5-Naphthalenedisulfonic acid (0.723 g, 2.509 mmol) in 5 ml of ethanol was added to a solution of 2-(isoindolin-2-ylmethyl)-5((1-(methylsulfonyl)piperidin-4-yl)methoxy)-4H-pyran-4-one (I) (2 g, 4.78 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h to yield 1,5-naphthalenedisulfonic acid salt of compound (I) (2.13 g, 79%). The product was analysed by XRPD and was found to be crystalline form 1 (Table 3). The X-ray powder diffraction pattern of form 1 is depicted in FIG. 5 and the differential scanning calorimetry (DSC) thermogram in FIG. 6. 1H NMR (400 MHz, DMSO-d6): δ ppm 1.21-1.37 (m, 2 H), 1.78-1.91 (m, 3 H), 2.66-2.76 (m, 2 H), 2.84-2.88 20 (m, 3 H), 3.53-3.63 (m, 2 H), 3.70-3.77 (m, 2 H), 4.55-4.75 (m, 4 H), 6.64 (s, 1 H), 7.28-7.40 (m, 4 H), 7.42 (s, 1 H), 7.91 (dd, 1 H), 8.23 (s, 1 H), 8.70-8.96 (m, 1 H), 10.88-11.54 (m, 1 H).

TABLE 3
X-ray powder reflections (up to 40° 2θ) and intensities
(normalized) of 1,5-naphthalenedisulfonic acid salt crystalline
form 1. The value 2θ [°] represents the diffraction
angle in degrees and the value d [Å] represents the
specified distances in Å between the lattice planes.

| 2θ [°] | d [Å] | I/Iₒ [%] |
|---|---|---|
| 5.88 | 15.01 | 3 |
| 9.18 | 9.62 | 3 |
| 10.63 | 8.32 | 20 |
| 11.73 | 7.54 | 4 |
| 12.39 | 7.14 | 2 |
| 13.73 | 6.44 | 8 |
| 13.97 | 6.33 | 1 |
| 14.15 | 6.25 | 4 |
| 14.63 | 6.05 | 4 |
| 14.75 | 6.00 | 4 |
| 15.16 | 5.84 | 4 |
| 15.45 | 5.73 | 18 |
| 16.04 | 5.52 | 11 |
| 16.23 | 5.46 | 4 |
| 16.59 | 5.34 | 7 |
| 17.05 | 5.20 | 31 |
| 17.63 | 5.03 | 100 |
| 18.38 | 4.82 | 17 |
| 19.50 | 4.55 | 34 |
| 20.15 | 4.40 | 48 |
| 20.41 | 4.35 | 80 |
| 20.56 | 4.32 | 24 |
| 21.06 | 4.21 | 20 |
| 21.38 | 4.15 | 14 |
| 21.99 | 4.04 | 5 |
| 22.40 | 3.97 | 9 |
| 22.80 | 3.90 | 45 |
| 23.32 | 3.81 | 10 |
| 23.84 | 3.73 | 45 |
| 24.35 | 3.65 | 8 |
| 24.83 | 3.58 | 67 |
| 25.12 | 3.54 | 6 |
| 25.23 | 3.53 | 10 |
| 25.51 | 3.49 | 23 |
| 26.37 | 3.38 | 8 |
| 26.85 | 3.32 | 6 |
| 27.05 | 3.29 | 4 |
| 27.40 | 3.25 | 21 |
| 27.66 | 3.22 | 32 |
| 27.96 | 3.19 | 6 |
| 28.38 | 3.14 | 9 |
| 28.75 | 3.10 | 20 |
| 29.40 | 3.04 | 8 |
| 30.12 | 2.97 | 27 |
| 30.51 | 2.93 | 3 |
| 30.93 | 2.89 | 4 |
| 31.74 | 2.82 | 27 |
| 32.33 | 2.77 | 1 |
| 33.25 | 2.69 | 4 |
| 33.49 | 2.67 | 2 |
| 33.77 | 2.65 | 2 |
| 34.44 | 2.60 | 3 |
| 35.13 | 2.55 | 8 |
| 35.49 | 2.53 | 4 |
| 35.88 | 2.50 | 4 |
| 36.76 | 2.44 | 3 |
| 37.15 | 2.42 | 7 |
| 37.57 | 2.39 | 9 |
| 38.03 | 2.36 | 3 |
| 38.25 | 2.35 | 3 |
| 38.58 | 2.33 | 5 |

### EXAMPLE 5

### Hydrobromic Acid Salt Crystalline Form 1

Fuming hydrobromic acid (0.690 ml, 5.97 mmol) in 5 ml of ethanol was added to a solution of 2-(isoindolin-2-ylmethyl)-5-((1-(methylsulfonyl)piperidin-4-yl)methoxy)-4H-pyran-4-one (I) (2 g, 4.78 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h to yield hydrobromic acid salt of compound (I) (2.2 g, 93%). The product was analysed by XRPD and was found to be crystalline form 1 (Table 4). The X-ray powder diffraction pattern of form 1 is depicted in FIG. 7 and the differential scanning calorimetry (DSC) thermogram in FIG. 8. 1H NMR (400 MHz, DMSO-d6): δ ppm 1.31 (br s, 2 H), 1.77-1.92 (m, 3 H), 2.67-2.77 (m, 2 H), 2.87 (s, 3 H), 3.59 (br d, 2 H), 3.75 (d, 2 H), 4.62 (br dd, 4 H), 6.64 (s, 1 H), 7.26-7.48 (m, 4 H), 8.24 (s, 1 H), 10.87-11.59 (m, 1 H).

TABLE 4
X-ray powder reflections (up to 40° 2θ) and intensities (normalized)
of hydrobromic acid salt crystalline form 1. The value 2θ [°]
represents the diffraction angle in degrees and the value d [Å]
represents the specified distances in Å between the lattice planes.

| 2θ [°] | d [Å] | I/Iₒ [%] |
|---|---|---|
| 5.28 | 16.71 | 9 |
| 8.87 | 9.96 | 1 |
| 10.47 | 8.44 | 81 |
| 12.75 | 6.94 | 3 |
| 13.43 | 6.59 | 5 |
| 13.59 | 6.51 | 17 |
| 14.54 | 6.09 | 5 |
| 15.43 | 5.74 | 5 |
| 15.72 | 5.63 | 6 |
| 16.93 | 5.23 | 55 |
| 17.20 | 5.15 | 55 |
| 17.32 | 5.12 | 36 |
| 17.94 | 4.94 | 2 |
| 18.34 | 4.83 | 73 |
| 18.75 | 4.73 | 51 |
| 19.27 | 4.60 | 3 |
| 19.61 | 4.52 | 2 |
| 20.62 | 4.30 | 19 |
| 20.93 | 4.24 | 66 |
| 21.24 | 4.18 | 65 |
| 21.38 | 4.15 | 100 |
| 21.51 | 4.13 | 39 |
| 21.91 | 4.05 | 50 |
| 22.59 | 3.93 | 85 |
| 22.94 | 3.87 | 44 |
| 23.44 | 3.79 | 28 |
| 23.66 | 3.76 | 32 |
| 24.10 | 3.69 | 27 |
| 24.45 | 3.64 | 16 |
| 25.82 | 3.45 | 34 |
| 26.21 | 3.40 | 2 |
| 26.82 | 3.32 | 28 |
| 26.93 | 3.31 | 64 |
| 27.26 | 3.27 | 30 |
| 27.37 | 3.26 | 15 |
| 27.79 | 3.21 | 19 |
| 28.76 | 3.10 | 23 |
| 28.87 | 3.09 | 11 |
| 29.17 | 3.06 | 13 |
| 29.62 | 3.01 | 20 |
| 30.06 | 2.97 | 13 |
| 30.43 | 2.94 | 23 |
| 30.77 | 2.90 | 8 |
| 31.18 | 2.87 | 11 |
| 31.40 | 2.85 | 37 |
| 31.77 | 2.81 | 8 |
| 32.17 | 2.78 | 19 |
| 33.16 | 2.70 | 15 |
| 33.53 | 2.67 | 11 |
| 34.35 | 2.61 | 13 |
| 34.83 | 2.57 | 1 |
| 35.44 | 2.53 | 8 |
| 35.85 | 2.50 | 5 |
| 36.22 | 2.48 | 7 |
| 37.03 | 2.43 | 24 |
| 37.72 | 2.38 | 29 |
| 38.24 | 2.35 | 10 |
| 38.61 | 2.33 | 10 |
| 39.42 | 2.28 | 2 |

### EXAMPLE 6

### Nitric Acid Salt Crystalline Form 1

Nitric acid (0.198 ml, 4.78 mmol) in 5 ml of ethanol was added to a solution of 2-(isoindolin-2-ylmethyl)-5-((1-(methylsulfonyl)piperidin-4-yl)methoxy)-4H-pyran-4-one (I) (2g, 4.78 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h to yield nitric acid salt of compound (I) (1.85 g, 80%). The product was analysed by XRPD and was found to be crystalline form 1. The X-ray powder diffraction pattern of form 1 is depicted in FIG. 9 and the differential scanning calorimetry (DSC) thermogram in FIG. 10. 1H NMR (400 MHz, DMSO-d6): δ ppm 1.21-1.37 (m, 2 H), 1.79-1.90 (m, 3 H), 2.68-2.77 (m, 2 H), 2.86 (s, 3 H), 3.54-3.68 (m, 2 H), 3.68-3.81 (m, 2 H), 4.46-4.70 (m, 4 H), 6.49-6.70 (m, 1 H), 7.38 (br d, 4 H), 8.24 (s, 1 H), 10.86-11.57 (m, 1 H).

TABLE 5
X-ray powder reflections (up to 40° 2θ) and intensities
(normalized) of nitric acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.37 | 16.46 | 2 |
| 8.82 | 10.02 | 4 |
| 10.70 | 8.26 | 80 |
| 10.78 | 8.20 | 28 |
| 12.81 | 6.91 | 5 |
| 13.22 | 6.69 | 2 |
| 13.45 | 6.58 | 16 |
| 13.98 | 6.33 | 11 |
| 14.99 | 5.90 | 13 |
| 16.04 | 5.52 | 20 |
| 16.33 | 5.42 | 13 |
| 16.74 | 5.29 | 6 |
| 16.95 | 5.23 | 50 |
| 17.29 | 5.13 | 73 |
| 17.57 | 5.04 | 48 |
| 17.93 | 4.94 | 82 |
| 18.15 | 4.88 | 10 |
| 18.39 | 4.82 | 57 |
| 19.44 | 4.56 | 3 |
| 20.29 | 4.37 | 54 |
| 20.75 | 4.28 | 72 |
| 21.15 | 4.20 | 44 |
| 21.39 | 4.15 | 82 |
| 21.82 | 4.07 | 58 |
| 22.07 | 4.02 | 100 |
| 22.65 | 3.92 | 59 |
| 23.04 | 3.86 | 37 |
| 23.51 | 3.78 | 33 |
| 24.07 | 3.69 | 13 |
| 24.68 | 3.60 | 3 |
| 25.13 | 3.54 | 16 |
| 25.72 | 3.46 | 17 |
| 26.20 | 3.40 | 11 |
| 26.51 | 3.36 | 38 |
| 26.97 | 3.30 | 11 |
| 27.25 | 3.27 | 28 |
| 27.88 | 3.20 | 21 |
| 28.13 | 3.17 | 25 |
| 28.67 | 3.11 | 10 |
| 28.90 | 3.09 | 9 |
| 30.09 | 2.97 | 2 |
| 30.48 | 2.93 | 14 |
| 30.77 | 2.90 | 6 |
| 31.08 | 2.87 | 2 |
| 31.84 | 2.81 | 4 |
| 32.20 | 2.78 | 9 |
| 32.64 | 2.74 | 30 |
| 33.02 | 2.71 | 7 |
| 33.39 | 2.68 | 5 |
| 33.75 | 2.65 | 3 |
| 34.19 | 2.62 | 7 |
| 34.65 | 2.59 | 4 |
| 35.57 | 2.52 | 4 |
| 35.96 | 2.50 | 11 |
| 36.65 | 2.45 | 11 |
| 37.11 | 2.42 | 8 |
| 37.72 | 2.38 | 4 |
| 38.09 | 2.36 | 3 |
| 38.75 | 2.32 | 12 |
| 39.07 | 2.30 | 10 |
| 39.51 | 2.28 | 4 |

### EXAMPLE 7

### Benzenesulfonic Acid Salt Crystalline Form 1

Benzenesulfonic acid (0.040 g, 0.260 mmol) in 0.2 ml of 2-propanol was added to a solution of 2-(isoindolin-2-ylmethyl)-5-((1-(methylsulfonyl)piperidin-4-yl)methoxy)-4H-pyran-4-one (I) (0.1 g, 0.239 mmol) in 0.8 ml of 2-propanol at refluxing temperature, and refluxed until all solids were dissolved. The mixture was stirred and allowed to cool to room temperature. The precipitated solid was filtered, washed twice with 2-propanol, and dried under vacuum at 40° C. for 16 h to yield benzenesulfonic acid salt of compound (I) (0.090 g, 65%). The product was analysed by XRPD and was found to be crystalline form 1. The X-ray powder diffraction pattern of form 1 is depicted in FIG. 11 and the differential scanning calorimetry (DSC) thermogram in FIG. 12. 1H NMR (400 MHz, DMSO-d6): δ ppm 1.19-1.38 (m, 2 H), 1.68-1.95 (m, 3 H), 2.64-2.74 (m, 2 H), 2.78-2.88 (m, 3 H), 3.57 (br s, 2 H), 3.65-3.78 (m, 2 H), 4.57-4.81 (m, 4 H), 6.49-6.73 (m, 1 H), 7.22-7.43 (m, 7 H), 7.48-7.63 (m, 2 H), 8.08-8.33 (m, 1 H), 11.02-11.51 (m, 1 H).

TABLE 6
X-ray powder reflections (up to 40° 2θ) and intensities
(normalized) of benzenesulfonic acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 4.58 | 19.27 | 100 |
| 9.13 | 9.68 | 67 |
| 11.68 | 7.57 | 7 |
| 13.69 | 6.46 | 33 |
| 14.76 | 6.00 | 1 |
| 15.51 | 5.71 | 10 |
| 16.25 | 5.45 | 1 |
| 16.60 | 5.34 | 1 |
| 16.94 | 5.23 | 1 |
| 17.21 | 5.15 | 2 |
| 17.36 | 5.10 | 1 |
| 17.83 | 4.97 | 1 |
| 18.28 | 4.85 | 4 |
| 18.50 | 4.79 | 2 |
| 19.19 | 4.62 | 1 |
| 19.69 | 4.51 | 37 |
| 20.11 | 4.41 | 1 |
| 20.88 | 4.25 | 2 |
| 21.29 | 4.17 | 1 |
| 21.88 | 4.06 | 2 |
| 22.44 | 3.96 | 2 |
| 22.88 | 3.88 | 11 |
| 23.50 | 3.78 | 2 |
| 24.04 | 3.70 | 12 |
| 25.16 | 3.54 | 1 |
| 26.12 | 3.41 | 1 |
| 26.31 | 3.38 | 0 |
| 26.72 | 3.33 | 1 |
| 27.52 | 3.24 | 16 |
| 28.51 | 3.13 | 5 |
| 29.22 | 3.05 | 1 |
| 29.98 | 2.98 | 1 |
| 30.36 | 2.94 | 1 |
| 30.72 | 2.91 | 0 |
| 31.30 | 2.86 | 2 |
| 33.06 | 2.71 | 4 |
| 33.55 | 2.67 | 1 |
| 34.07 | 2.63 | 1 |
| 34.97 | 2.56 | 0 |
| 35.53 | 2.52 | 2 |
| 37.13 | 2.42 | 1 |
| 37.73 | 2.38 | 2 |
| 38.62 | 2.33 | 1 |

### EXAMPLE 8

### Hydrochloric Acid Salt Crystalline Form 1

Hydrochloric acid (1M in diethyl ether, 0.358 ml, 0.358 mmol) was added to a solution of 2-(isoindolin-2-ylmethyl)-5-((1-(methylsulfonyl)piperidin-4-yl)-methoxy)-4H-pyran-4-one (I) (0.15 g, 0.358 mmol) in 7.5 ml of 2-propanol at 80° C. followed by heating. The mixture was stirred and allowed to cool to room temperature. The precipitated solid was filtered and dried under vacuum at 40° C. for 16 h to yield hydrochloric acid salt of compound (I) (0.14 g, 87%). The product was analysed by XRPD and was found to be crystalline form 1. The X-ray powder diffraction pattern of form 1 is depicted in FIG. 13. 1H NMR (400 MHz, DMSO-d6): δ ppm 1.22-1.38 (m, 2 H), 1.78-1.93 (m, 3 H), 2.66-2.78 (m, 2 H), 2.86 (s, 3

H), 3.53-3.64 (m, 2 H), 3.71-3.80 (m, 2 H), 4.58-4.81 (m, 4 H), 6.72 (s, 1 H), 7.20-7.49 (m, 4 H), 8.23 (s, 1 H), 11.37-13.72 (m, 1 H).

TABLE 7
X-ray powder reflections (up to 40° 2θ) and intensities
(normalized) of hydrochloric acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 4.7 | 19.0 | 26 |
| 9.3 | 9.5 | 75 |
| 9.6 | 9.2 | 17 |
| 13.9 | 6.3 | 46 |
| 14.4 | 6.2 | 18 |
| 16.0 | 5.5 | 100 |
| 16.6 | 5.3 | 63 |
| 17.2 | 5.1 | 44 |
| 17.8 | 5.0 | 57 |
| 18.4 | 4.8 | 13 |
| 19.5 | 4.5 | 21 |
| 19.9 | 4.5 | 32 |
| 20.1 | 4.4 | 55 |
| 21.1 | 4.2 | 70 |
| 21.4 | 4.1 | 52 |
| 22.2 | 4.0 | 67 |
| 22.9 | 3.9 | 40 |
| 23.3 | 3.8 | 76 |
| 23.8 | 3.7 | 46 |
| 24.7 | 3.6 | 39 |
| 26.8 | 3.3 | 26 |
| 29.1 | 3.1 | 9 |
| 30.4 | 2.9 | 13 |
| 32.7 | 2.7 | 8 |

### EXAMPLE 9

### Hydrochloric Acid Salt Crystalline Form 2

Hydrochloric acid (3M in cyclopentylmethylether, 0.219 ml, 0.657 mmol) was added to a solution of 2-(isoindolin-2-ylmethyl)-5-((1-(methylsulfonyl)piperidin-4-yl)methoxy)-4H-pyran-4-one (I) (0.22 g, 0.526 mmol) in 4 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h. Recrystallization of the product was then carried out by dissolving the solid to 0.9 ml of 2-propanol:water 5:4 under heating followed by cooling in ice bath. The obtained precipitate was isolated by filtering, was washed with isopropanol and dried as above to yield hydrochloric acid salt of compound (I) (0.11 g, 46%). The product was analysed by XRPD and was found to be crystalline form 2. The X-ray powder diffraction pattern of form 2 is depicted in FIG. 14. 1H NMR (400 MHz, DMSO-d6): δ ppm 1.22-1.38 (m, 2 H), 1.78-1.93 (m, 3 H), 2.66-2.78 (m, 2 H), 2.86 (s, 3 H), 3.53-3.64 (m, 2 H), 3.71-3.80 (m, 2 H), 4.58-4.81 (m, 4 H), 6.72 (s, 1 H), 7.20-7.49 (m, 4 H), 8.23 (s, 1 H), 11.37-13.72 (m, 1 H).

TABLE 8
X-ray powder reflections (up to 40° 2θ) and intensities
(normalized) of hydrochloric acid salt crystalline form 2.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 4.85 | 18.19 | 51 |
| 7.26 | 12.17 | 100 |
| 9.67 | 9.14 | 10 |
| 12.08 | 7.32 | 0 |
| 14.51 | 6.10 | 43 |
| 16.93 | 5.23 | 46 |
| 17.71 | 5.00 | 1 |
| 18.12 | 4.89 | 2 |
| 18.78 | 4.72 | 3 |
| 19.35 | 4.58 | 4 |
| 19.74 | 4.49 | 3 |
| 21.44 | 4.14 | 1 |
| 21.80 | 4.07 | 1 |
| 22.32 | 3.98 | 4 |
| 22.86 | 3.89 | 1 |
| 23.56 | 3.77 | 1 |
| 24.26 | 3.67 | 60 |
| 25.05 | 3.55 | 2 |
| 26.97 | 3.30 | 2 |
| 28.99 | 3.08 | 3 |
| 29.38 | 3.04 | 2 |
| 30.06 | 2.97 | 0 |
| 31.69 | 2.82 | 4 |
| 33.20 | 2.70 | 0 |
| 35.13 | 2.55 | 0 |
| 36.73 | 2.45 | 2 |
| 37.82 | 2.38 | 1 |
| 39.27 | 2.29 | 4 |

### EXAMPLE 10

### Hydrochloric Acid Salt Crystalline Form 3

Hydrochloric acid (6 M, 1.374 ml, 8.24 mmol) was added to a solution of 2-(isoindolin-2-ylmethyl)-5-((1-(methylsulfonyl)piperidin-4-yl)methoxy)-4H-pyran-4-one (I) (3.0 g, 0.717 mmol) in 15 ml of ethanol at refluxing temperature, and refluxed for 5 minutes. The mixture was stirred and allowed to cool to room temperature. The precipitated solid was filtered, washed with ethanol, and dried under vacuum at 40° C. for 16 h. This product was then dissolved in 35 ml of EtOH:water 5:1 at refluxing temperature under stirring for 10 min. The mixture was allowed to cool. The obtained solid was isolated by filtering and dried under vacuum at 40° C. for 16 h to yield hydrochloric acid salt of compound (I) (2.0 g, 61%). The product was analysed by XRPD and was found to be crystalline form 3. The X-ray powder diffraction pattern of form 3 is depicted in FIG. 15 and the differential scanning calorimetry (DSC) thermogram in FIG. 16. 1H NMR (400 MHz, DMSO-d6): δ ppm 1.22-1.38 (m, 2 H), 1.78-1.93 (m, 3 H), 2.66-2.78 (m, 2 H), 2.86 (s, 3 H), 3.53-3.64 (m, 2 H), 3.71-3.80 (m, 2 H), 4.58-4.81 (m, 4 H), 6.72 (s, 1 H), 7.20 -7.49 (m, 4 H), 8.23 (s, 1 H), 11.37-13.72 (m, 1 H).

TABLE 9
X-ray powder reflections (up to 40° 2θ) and intensities
(normalized) of hydrochloric acid salt crystalline form 3.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 10.5 | 8.4 | 5 |
| 10.6 | 8.3 | 3 |
| 13.6 | 6.5 | 1 |
| 14.4 | 6.1 | 2 |
| 14.8 | 6.0 | 2 |
| 15.8 | 5.6 | 100 |
| 15.9 | 5.6 | 94 |
| 16.9 | 5.2 | 10 |
| 17.8 | 5.0 | 8 |
| 18.1 | 4.9 | 3 |
| 18.4 | 4.8 | 2 |
| 19.1 | 4.7 | 20 |
| 19.2 | 4.6 | 12 |
| 20.1 | 4.4 | 9 |
| 20.2 | 4.4 | 14 |
| 20.6 | 4.3 | 3 |
| 21.0 | 4.2 | 45 |
| 21.1 | 4.2 | 6 |
| 22.5 | 4.0 | 50 |
| 22.6 | 3.9 | 26 |
| 23.9 | 3.7 | 27 |
| 25.1 | 3.5 | 6 |
| 25.8 | 3.5 | 3 |
| 26.3 | 3.4 | 8 |
| 26.8 | 3.3 | 12 |
| 27.1 | 3.3 | 3 |
| 27.4 | 3.3 | 7 |
| 28.6 | 3.1 | 3 |
| 28.9 | 3.1 | 3 |
| 29.8 | 3.0 | 22 |
| 30.9 | 2.9 | 1 |
| 31.4 | 2.8 | 2 |
| 31.7 | 2.8 | 7 |
| 31.8 | 2.8 | 9 |
| 32.5 | 2.8 | 22 |
| 34.1 | 2.6 | 20 |
| 34.7 | 2.6 | 2 |
| 35.8 | 2.5 | 13 |
| 35.9 | 2.5 | 3 |
| 36.9 | 2.4 | 1 |
| 37.3 | 2.4 | 0 |
| 37.4 | 2.4 | 2 |
| 37.7 | 2.4 | 1 |
| 39.4 | 2.3 | 11 |
| 39.5 | 2.3 | 6 |
| 39.9 | 2.3 | 0 |

### EXAMPLE 11

### Maleic Acid Salt Crystalline Form 1

The mixture of maleic acid (0.014 g, 0.119 mmol) and 2-(isoindolin-2-yl-methyl)-5-((1-(methylsulfonyl)piperidin-4-yl)methoxy)-4H-pyran-4-one (I) (0.050 g, 0.119 mmol) was heated in 2.5 ml of 2-propanol at 80° C. The mixture was stirred and allowed to cool to room temperature. The precipitated solid was filtered, washed twice with 2-propanol and dried under vacuum at 40° C. for 16 h to yield maleic acid salt of compound (I) (0.049 g, 77%). The product was analysed by XRPD and was found to be crystalline form 1. The X-ray powder diffraction pattern of form 1 is depicted in FIG. 17. 1H NMR (400 MHz, DMSO-d6): δ ppm 1.24-1.36 (m, 2 H), 1.79-1.90 (m, 3 H), 2.69-2.77 (m, 2 H), 2.86 (s, 3 H), 3.56-3.61 (m, 2 H), 3.71 -3.81 (m, 2 H), 4.07-4.10 (m, 1 H), 4.15-4.32 (m, 4 H), 6.18 (s, 2 H), 6.41-6.54 (m, 1 H), 7.22-7.35 (m, 4 H), 8.14-8.24 (m, 1 H).

TABLE 10
X-ray powder reflections (up to 40° 2θ) and intensities
(normalized) of maleic acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.5 | 16.2 | 12 |
| 6.1 | 14.6 | 2 |
| 9.0 | 9.8 | 4 |
| 10.9 | 8.1 | 30 |
| 11.2 | 7.9 | 18 |
| 12.1 | 7.3 | 15 |
| 13.7 | 6.5 | 40 |
| 14.3 | 6.2 | 1 |
| 16.1 | 5.5 | 10 |
| 16.4 | 5.4 | 63 |
| 17.3 | 5.1 | 2 |
| 17.7 | 5.0 | 50 |
| 18.0 | 4.9 | 11 |
| 18.1 | 4.9 | 9 |
| 18.8 | 4.7 | 50 |
| 19.0 | 4.7 | 7 |
| 19.6 | 4.5 | 48 |
| 20.2 | 4.4 | 21 |
| 20.4 | 4.3 | 18 |
| 20.7 | 4.3 | 9 |
| 21.3 | 4.2 | 100 |
| 21.9 | 4.1 | 99 |
| 22.4 | 4.0 | 29 |
| 23.1 | 3.9 | 4 |
| 23.9 | 3.7 | 4 |
| 24.5 | 3.6 | 10 |
| 26.5 | 3.4 | 6 |
| 26.7 | 3.3 | 6 |
| 27.0 | 3.3 | 9 |
| 27.3 | 3.3 | 12 |
| 27.5 | 3.2 | 7 |
| 28.2 | 3.2 | 5 |
| 28.5 | 3.1 | 18 |
| 28.8 | 3.1 | 8 |
| 29.5 | 3.0 | 3 |
| 29.7 | 3.0 | 0 |
| 31.7 | 2.8 | 8 |
| 32.1 | 2.8 | 9 |
| 32.2 | 2.8 | 7 |
| 34.0 | 2.6 | 5 |
| 35.4 | 2.5 | 4 |
| 36.4 | 2.5 | 5 |
| 36.6 | 2.5 | 4 |
| 37.9 | 2.4 | 1 |
| 38.8 | 2.3 | 10 |

### EXAMPLE 12

### Maleic Acid Salt Crystalline Form 2

Maleic acid (0.139 g, 1.195 mmol) was added to a solution of 2-(isoindolin-2-ylmethyl)-5-((1-(methylsulfonyl)piperidin-4-yl)methoxy)-4H-pyran-4-one (I) (0.5 g, 1.195 mmol) in ethanol (8 ml) at refluxing temperature, and refluxed until all solids were dissolved. The mixture was stirred and allowed to cool to room temperature. The precipitated solid was filtered, washed twice with ethanol, and dried under vacuum at 40° C. for 16 h to yield maleic acid salt of compound (I) (0.61 g, 95%).

The product was analysed by XRPD and was found to be crystalline form 2. The X-ray powder diffraction pattern of form 2 is depicted in FIG. 18. 1H NMR (400 MHz, DMSO-d6): δ ppm 1.24-1.36 (m, 2 H), 1.79-1.90 (m, 3 H), 2.69-2.77 (m, 2 H), 2.86 (s, 3 H), 3.56-3.61 (m, 2 H), 3.71-3.81 (m, 2 H), 4.07-4.10 (m, 1 H), 4.15-4.32 (m, 4 H), 6.18 (s, 2 H), 6.41-6.54 (m, 1 H), 7.22-7.35 (m, 4 H), 8.14 -8.24 (m, 1 H).

TABLE 11
X-ray powder reflections (up to 40° 2θ) and intensities
(normalized) of maleic acid salt crystalline form 2. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.06 | 17.44 | 2 |
| 6.88 | 12.84 | 1 |
| 7.43 | 11.90 | 27 |
| 9.49 | 9.31 | 2 |
| 10.72 | 8.24 | 38 |
| 13.39 | 6.61 | 27 |
| 14.90 | 5.94 | 51 |
| 15.27 | 5.80 | 4 |
| 15.79 | 5.61 | 7 |
| 17.00 | 5.21 | 18 |
| 17.37 | 5.10 | 19 |
| 18.25 | 4.86 | 39 |
| 18.97 | 4.68 | 17 |
| 19.38 | 4.58 | 45 |
| 20.38 | 4.35 | 17 |
| 20.71 | 4.29 | 15 |
| 21.40 | 4.15 | 9 |
| 21.59 | 4.11 | 17 |
| 22.44 | 3.96 | 100 |
| 23.03 | 3.86 | 4 |
| 23.41 | 3.80 | 6 |
| 23.75 | 3.74 | 10 |
| 24.09 | 3.69 | 8 |
| 24.71 | 3.60 | 10 |
| 25.19 | 3.53 | 18 |
| 26.17 | 3.40 | 4 |
| 26.96 | 3.30 | 10 |
| 27.45 | 3.25 | 6 |
| 28.60 | 3.12 | 8 |
| 30.07 | 2.97 | 7 |
| 31.20 | 2.86 | 3 |
| 32.77 | 2.73 | 10 |

### EXAMPLE 13

### Maleic Acid Salt Crystalline Form 3

Maleic acid (0.832g, 0.717 mmol) in 15 ml of ethanol was added to a solution of 2-(isoindolin-2-ylmethyl)-5-((1-(methylsulfonyl)piperidin-4-yl)methoxy)-4H-pyran-4-one (I) (3 g, 0.717 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h. This product was stirred in 36 ml of EtOH:water 33:5 at the refluxing temperature until all solids were dissolved. The mixture was allowed to cool. The obtained solid was isolated by filtering and dried under vacuum at 40° C. for 16 h to yield maleic acid salt of compound (I) (2.48 g, 65%). The product was analysed by XRPD and was found to be crystalline form 3. The X-ray powder diffraction pattern of form 3 is depicted in FIG. 19 and the differential scanning calorimetry (DSC) thermogram in FIG. 20. 1H NMR (400 MHz, DMSO-d6): δ ppm 1.24-1.36 (m, 2 H), 1.79-1.90 (m, 3 H), 2.69-2.77 (m, 2 H), 2.86 (s, 3 H), 3.56-3.61 (m, 2 H), 3.71-3.81 (m, 2 H), 4.07-4.10 (m, 1 H), 4.15-4.32 (m, 4 H), 6.18 (s, 2 H), 6.41-6.54 (m, 1 H), 7.22-7.35 (m, 4 H), 8.14 -8.24 (m, 1 H).

TABLE 12
X-ray powder reflections (up to 40° 2θ) and intensities
(normalized) of maleic acid salt crystalline form 3. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.88 | 15.03 | 8 |
| 9.27 | 9.53 | 4 |
| 11.27 | 7.85 | 9 |
| 11.44 | 7.73 | 24 |
| 11.78 | 7.50 | 15 |
| 14.51 | 6.10 | 29 |
| 16.17 | 5.48 | 42 |
| 16.71 | 5.30 | 5 |
| 16.91 | 5.24 | 23 |
| 17.54 | 5.05 | 5 |
| 17.72 | 5.00 | 52 |
| 17.94 | 4.94 | 17 |
| 18.82 | 4.71 | 20 |
| 19.23 | 4.61 | 21 |
| 19.56 | 4.54 | 9 |
| 20.17 | 4.40 | 7 |
| 20.56 | 4.32 | 31 |
| 21.32 | 4.16 | 14 |
| 21.60 | 4.11 | 5 |
| 21.99 | 4.04 | 8 |
| 22.66 | 3.92 | 50 |
| 23.00 | 3.86 | 100 |
| 23.60 | 3.77 | 31 |
| 23.71 | 3.75 | 85 |
| 24.19 | 3.68 | 6 |
| 24.90 | 3.57 | 5 |
| 25.57 | 3.48 | 7 |
| 25.97 | 3.43 | 18 |
| 26.61 | 3.35 | 11 |
| 26.96 | 3.30 | 11 |
| 27.26 | 3.27 | 5 |
| 27.62 | 3.23 | 3 |
| 28.09 | 3.17 | 3 |
| 28.48 | 3.13 | 24 |
| 28.95 | 3.08 | 2 |
| 29.24 | 3.05 | 1 |
| 29.75 | 3.00 | 3 |
| 30.38 | 2.94 | 4 |
| 30.85 | 2.90 | 2 |
| 31.19 | 2.87 | 3 |
| 31.97 | 2.80 | 3 |
| 32.32 | 2.77 | 2 |
| 32.71 | 2.74 | 5 |
| 33.07 | 2.71 | 6 |
| 34.34 | 2.61 | 0 |
| 34.77 | 2.58 | 3 |
| 35.49 | 2.53 | 4 |
| 36.14 | 2.48 | 7 |

### EXAMPLE 14

### 1,2-Ethanedisulfonic Acid Salt Crystalline Form 1

1,2-Ethanedisulfonic acid (0.389 g, 2.044 mmol) in 5 ml of ethanol was added to a solution of 2-(isoindolin-2-ylmethyl)-5-((1-(methylsulfonyl)piperidin-4-yl)-methoxy)-4H-pyran-4-one (I) (2 g, 3.89 mmol) in 15 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h to yield 1,2-ethanedisulfonic acid salt of compound (I) (2.07 g, 104%). The product was analysed by XRPD and was found to be crystalline form 1. The X-ray powder diffraction pattern of form 1 is depicted in FIG. 21 and the differential scanning calorimetry (DSC) thermogram in FIG. 22. 1H NMR (400 MHz, DMSO-d6): δ ppm 1.21-1.37 (m, 2 H), 1.85 (br d, 3 H), 2.63 (s, 2 H), 2.73 (td, 2 H), 2.87 (s, 3 H), 3.59 (br d, 2 H), 3.75 (d, 2 H), 4.59-4.86 (m, 4 H), 6.65 (s, 1 H), 7.18-7.49 (m, 4 H), 8.26 (s, 1 H), 10.90 -11.63 (m, 1 H).

TABLE 13
X-ray powder reflections (up to 40° 2θ) and intensities (normalized)
of 1,2-ethanedisulfonic acid salt crystalline form 1. The value 2θ
[°] represents the diffraction angle in degrees and the value
d [Å] represents the specified distances in Å between the lattice planes.

| 2θ [°] | d [Å] | I/Iₒ [%] |
|---|---|---|
| 7.92 | 11.16 | 17 |
| 9.07 | 9.74 | 36 |
| 10.72 | 8.25 | 13 |
| 14.92 | 5.93 | 20 |
| 15.16 | 5.84 | 14 |
| 15.78 | 5.61 | 1 |
| 15.98 | 5.54 | 1 |
| 16.79 | 5.28 | 16 |
| 17.10 | 5.18 | 1 |
| 18.00 | 4.92 | 2 |
| 18.13 | 4.89 | 3 |
| 19.43 | 4.56 | 9 |
| 19.69 | 4.51 | 3 |
| 20.22 | 4.39 | 2 |
| 20.50 | 4.33 | 10 |
| 21.11 | 4.21 | 4 |
| 21.44 | 4.14 | 1 |
| 21.84 | 4.07 | 2 |
| 22.01 | 4.04 | 2 |
| 22.60 | 3.93 | 2 |
| 23.14 | 3.84 | 1 |
| 23.70 | 3.75 | 100 |
| 24.28 | 3.66 | 5 |
| 24.68 | 3.61 | 7 |
| 25.69 | 3.46 | 3 |
| 26.28 | 3.39 | 1 |
| 26.53 | 3.36 | 6 |
| 27.25 | 3.27 | 12 |
| 27.69 | 3.22 | 2 |
| 28.10 | 3.17 | 1 |
| 28.64 | 3.11 | 3 |
| 28.81 | 3.10 | 2 |
| 29.78 | 3.00 | 4 |
| 30.10 | 2.97 | 2 |
| 30.99 | 2.88 | 10 |
| 31.33 | 2.85 | 1 |
| 31.76 | 2.82 | 7 |
| 32.38 | 2.76 | 3 |
| 33.04 | 2.71 | 1 |
| 34.30 | 2.61 | 3 |
| 35.01 | 2.56 | 0 |
| 35.94 | 2.50 | 1 |
| 36.12 | 2.48 | 1 |
| 36.58 | 2.45 | 3 |
| 37.02 | 2.43 | 1 |

### EXAMPLE 15

### Oxalic Acid Salt Crystalline Form 1

The mixture of oxalic acid (0.022 g, 0.239 mmol) and 2-(isoindolin-2-yl-methyl)-5-((1-(methylsulfonyl)piperidin-4-yl)methoxy)-4H-pyran-4-one (I) (0.1 g, 0.239 mmol) was refluxed in ethanol (1 ml) until all solids were dissolved. The mixture was stirred and allowed to cool to room temperature. The precipitated solid was filtered, washed twice with ethanol, and dried under vacuum at 40° C. for 16 h to yield oxalic acid salt of compound (I) (0.063 g, 52%). The product was analysed by XRPD and was found to be crystalline form 1. The X-ray powder diffraction pattern of form 1 is depicted in FIG. 23 and the differential scanning calorimetry (DSC) thermogram in FIG. 24. 1H NMR (400 MHz, DMSO-d6): δ ppm 1.22-1.35 (m, 2 H), 1.70-1.96 (m, 3 H), 2.68-2.76 (m, 2 H), 2.86 (s, 3 H), 3.50-3.60 (m, 2 H), 3.63-3.74 (m, 2 H), 3.89-3.90 (m, 1 H), 4.05 (s, 4 H), 6.35-6.50 (m, 1 H), 7.08 -7.34 (m, 4 H), 8.02-8.24 (m, 1 H).

TABLE 14
X-ray powder reflections (up to 40° 2θ) and intensities
(normalized) of oxalic acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 6.07 | 14.55 | 24 |
| 6.60 | 13.39 | 11 |
| 10.86 | 8.14 | 5 |
| 11.73 | 7.54 | 25 |
| 12.20 | 7.25 | 4 |
| 13.23 | 6.69 | 19 |
| 13.55 | 6.53 | 6 |
| 14.90 | 5.94 | 2 |
| 16.41 | 5.40 | 5 |
| 16.96 | 5.22 | 22 |
| 17.90 | 4.95 | 9 |
| 18.69 | 4.74 | 100 |
| 19.29 | 4.60 | 48 |
| 19.91 | 4.46 | 17 |
| 20.57 | 4.32 | 13 |
| 21.48 | 4.13 | 2 |
| 22.16 | 4.01 | 25 |
| 22.53 | 3.94 | 21 |
| 23.41 | 3.80 | 17 |
| 23.81 | 3.73 | 18 |
| 24.56 | 3.62 | 13 |
| 24.76 | 3.59 | 7 |
| 25.22 | 3.53 | 29 |
| 25.57 | 3.48 | 4 |
| 26.14 | 3.41 | 10 |
| 26.51 | 3.36 | 11 |
| 27.21 | 3.28 | 8 |
| 27.98 | 3.19 | 1 |
| 28.75 | 3.10 | 5 |
| 29.81 | 2.99 | 1 |
| 30.40 | 2.94 | 6 |
| 30.88 | 2.89 | 2 |
| 31.37 | 2.85 | 2 |
| 32.22 | 2.78 | 6 |
| 33.05 | 2.71 | 2 |
| 33.74 | 2.65 | 2 |
| 34.29 | 2.61 | 4 |
| 35.06 | 2.56 | 1 |
| 35.61 | 2.52 | 5 |

### EXAMPLE 16

### Oxalic Acid Salt Crystalline Form 2

Oxalic acid (0.022 g, 0.239 mmol) was added to a solution of 2-(isoindolin-2-ylmethyl)-5-((1-(methylsulfonyl)piperidin-4-yl)methoxy)-4H-pyran-4-one (I) (0.1g, 0.239 mmol) in 1 ml of acetonitrile at refluxing temperature, and refluxed until all solids were dissolved. The mixture was stirred and allowed to cool to room temperature. The precipitated solid was filtered, washed twice with acetonitrile, and dried under vacuum at 40° C. for 16 h to yield oxalic acid salt of compound (I) (0.087 g, 72%). The product was analysed by XRPD and was found to be crystalline form 2. The X-ray powder diffraction pattern of form 2 is depicted in FIG. 25. 1H NMR (400 MHz, DMSO-d6): δ ppm 1.22-1.35 (m, 2 H), 1.70-1.96 (m, 3 H), 2.68-2.76 (m, 2 H), 2.86 (s, 3 H), 3.50-3.60 (m, 2 H), 3.63-3.74 (m, 2 H), 3.89-3.90 (m, 1 H), 4.05 (s, 4 H), 6.35-6.50 (m, 1 H), 7.08-7.34 (m, 4 H), 8.02-8.24 (m, 1 H).

TABLE 15
X-ray powder reflections (up to 40° 2θ) and intensities
(normalized) of oxalic acid salt crystalline form 2. The value
2θ [°] represents the diffraction angle in degrees
and the value d [Å] represents the specified distances
in Å between the lattice planes.

| 2θ [°] | d [Å] | I/Iₒ [%] |
|---|---|---|
| 4.18 | 21.12 | 100 |
| 5.38 | 16.42 | 81 |
| 6.36 | 13.89 | 54 |
| 6.47 | 13.66 | 40 |
| 6.99 | 12.64 | 8 |
| 8.55 | 10.34 | 22 |
| 10.34 | 8.55 | 11 |
| 11.08 | 7.98 | 65 |
| 13.92 | 6.36 | 13 |
| 14.52 | 6.10 | 13 |
| 15.27 | 5.80 | 52 |
| 16.94 | 5.23 | 45 |
| 18.08 | 4.90 | 48 |
| 18.69 | 4.74 | 42 |
| 18.91 | 4.69 | 63 |
| 19.47 | 4.56 | 96 |
| 20.20 | 4.39 | 51 |
| 20.60 | 4.31 | 38 |
| 20.89 | 4.25 | 48 |
| 21.24 | 4.18 | 35 |
| 22.20 | 4.00 | 38 |
| 22.98 | 3.87 | 27 |
| 24.01 | 3.70 | 22 |
| 25.16 | 3.54 | 21 |
| 26.39 | 3.37 | 11 |
| 27.65 | 3.22 | 8 |
| 29.99 | 2.98 | 5 |
| 33.75 | 2.65 | 5 |

### EXAMPLE 17

### Ethanesulfonic Acid Salt Crystalline Form 1

Ethanesulfonic acid (0.585 ml, 7.17 mmol) in 5 ml of ethanol was added to a solution of 2-(isoindolin-2-ylmethyl)-5-((1-(methylsulfonyl)piperidin-4-yl)methoxy)-4H-pyran-4-one (I) (3 g, 7.17 mmol) in 15 ml of ethanol at refluxing temperature. The solvent was removed in vacuo, followed by recrystallization in 96% ethanol to yield ethanesulfonic acid salt of compound (I) (1.31 g, 34%). The product was analysed by XRPD and was found to be crystalline form 1. The X-ray powder diffraction pattern of form 1 is depicted in FIG. 26. 1H NMR (400 MHz, DMSO-d6): δ ppm 1.04 (t, 3 H), 1.23-1.38 (m, 2 H), 1.76-1.92 (m, 3 H), 2.32-2.40 (m, 2 H), 2.70-2.80 (m, 2 H), 2.87 (s, 3 H), 3.54-3.61 (m, 2 H), 3.68-3.78 (m, 2 H), 4.59- 4.83 (m, 4 H), 6.55-6.73 (m, 1 H), 7.31-7.46 (m, 4 H), 8.25 (s, 1 H), 11.00-11.64 (m, 1 H).

TABLE 16
X-ray powder reflections (up to 40° 2θ) and intensities
(normalized) of ethanesulfonic acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 5.22 | 16.92 | 13 |
| 8.38 | 10.55 | 3 |
| 8.48 | 10.42 | 2 |
| 10.41 | 8.49 | 100 |
| 11.61 | 7.62 | 1 |
| 12.31 | 7.19 | 31 |
| 13.19 | 6.71 | 4 |
| 14.66 | 6.04 | 3 |
| 15.59 | 5.68 | 87 |
| 15.99 | 5.54 | 5 |
| 16.26 | 5.45 | 18 |
| 16.74 | 5.29 | 7 |
| 16.84 | 5.26 | 11 |
| 17.99 | 4.93 | 2 |
| 18.52 | 4.79 | 24 |
| 18.65 | 4.75 | 89 |
| 18.78 | 4.72 | 17 |
| 20.17 | 4.40 | 6 |
| 20.45 | 4.34 | 18 |
| 20.76 | 4.27 | 22 |
| 21.35 | 4.16 | 34 |
| 21.91 | 4.05 | 14 |
| 22.33 | 3.98 | 10 |
| 23.23 | 3.83 | 25 |
| 23.75 | 3.74 | 19 |
| 24.65 | 3.61 | 4 |
| 25.25 | 3.52 | 5 |
| 25.44 | 3.50 | 5 |
| 26.01 | 3.42 | 28 |
| 26.45 | 3.37 | 9 |
| 26.91 | 3.31 | 23 |
| 27.57 | 3.23 | 3 |
| 28.07 | 3.18 | 34 |
| 29.33 | 3.04 | 2 |
| 29.91 | 2.99 | 8 |
| 30.63 | 2.92 | 9 |
| 31.35 | 2.85 | 11 |
| 31.65 | 2.83 | 3 |
| 32.60 | 2.74 | 5 |
| 32.98 | 2.71 | 7 |
| 33.48 | 2.67 | 3 |
| 35.03 | 2.56 | 10 |

### EXAMPLE 18

### Sulfuric Acid Salt Crystalline Form 1

Sulfuric acid (0.023 g, 0.239 mmol) in 0.2 ml of ethanol was added to a solution of 2-(isoindolin-2-ylmethyl)-5-((1-(methylsulfonyl)piperidin-4-yl)methoxy)-4H-pyran-4-one (I) (0.1g, 0.239 mmol) in 0.8 ml of acetonitrile at refluxing temperature, and refluxed until all solids were dissolved. The mixture was stirred and allowed to cool to room temperature. The precipitated solid was filtered, washed twice with acetonitrile, and dried under vacuum at 40° C. for 16 h to yield sulfuric acid salt of compound (I) (0.070 g, 57%). The product was analysed by XRPD and was found to be crystalline form 1. The X-ray powder diffraction pattern of form 1 is depicted in FIG. 27. 1H NMR (400 MHz, DMSO-d6): δ ppm 1.21-1.38 (m, 2 H), 1.86 (br s, 3 H), 2.73 (br d, 2 H), 2.86 (s, 3 H), 3.59 (br d, 2 H), 3.75 (d, 2 H), 4.60 -4.80 (m, 4 H), 6.65 (s, 1 H), 7.39 (br d, 4 H), 8.24 (s, 1 H), 10.70-11.78 (m, 1 H).

TABLE 17
X-ray powder reflections (up to 40° 2θ) and intensities
(normalized) of sulfuric acid salt crystalline form 1. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 5.53 | 15.97 | 33 |
| 8.54 | 10.35 | 2 |
| 11.05 | 8.00 | 100 |
| 13.04 | 6.79 | 11 |
| 13.37 | 6.62 | 3 |
| 14.14 | 6.26 | 0 |
| 15.49 | 5.72 | 9 |
| 15.88 | 5.57 | 6 |
| 17.03 | 5.20 | 37 |
| 17.27 | 5.13 | 48 |
| 17.45 | 5.08 | 71 |
| 17.84 | 4.97 | 25 |
| 18.21 | 4.87 | 16 |
| 20.17 | 4.40 | 13 |
| 20.59 | 4.31 | 16 |
| 20.77 | 4.27 | 50 |
| 21.14 | 4.20 | 36 |
| 21.31 | 4.17 | 58 |
| 21.47 | 4.14 | 29 |
| 21.73 | 4.09 | 14 |
| 22.18 | 4.00 | 43 |
| 23.42 | 3.79 | 24 |
| 23.60 | 3.77 | 13 |
| 24.89 | 3.57 | 8 |
| 25.28 | 3.52 | 10 |
| 25.80 | 3.45 | 13 |
| 26.43 | 3.37 | 5 |
| 26.80 | 3.32 | 14 |
| 27.50 | 3.24 | 4 |
| 27.81 | 3.21 | 10 |
| 28.59 | 3.12 | 10 |
| 30.33 | 2.94 | 2 |
| 30.80 | 2.90 | 4 |
| 31.16 | 2.87 | 6 |
| 32.65 | 2.74 | 4 |
| 33.30 | 2.69 | 9 |
| 34.20 | 2.62 | 5 |
| 35.79 | 2.51 | 1 |
| 36.75 | 2.44 | 2 |
| 37.25 | 2.41 | 1 |

### EXAMPLE 19

### Sulfuric Acid Salt Crystalline Form 2

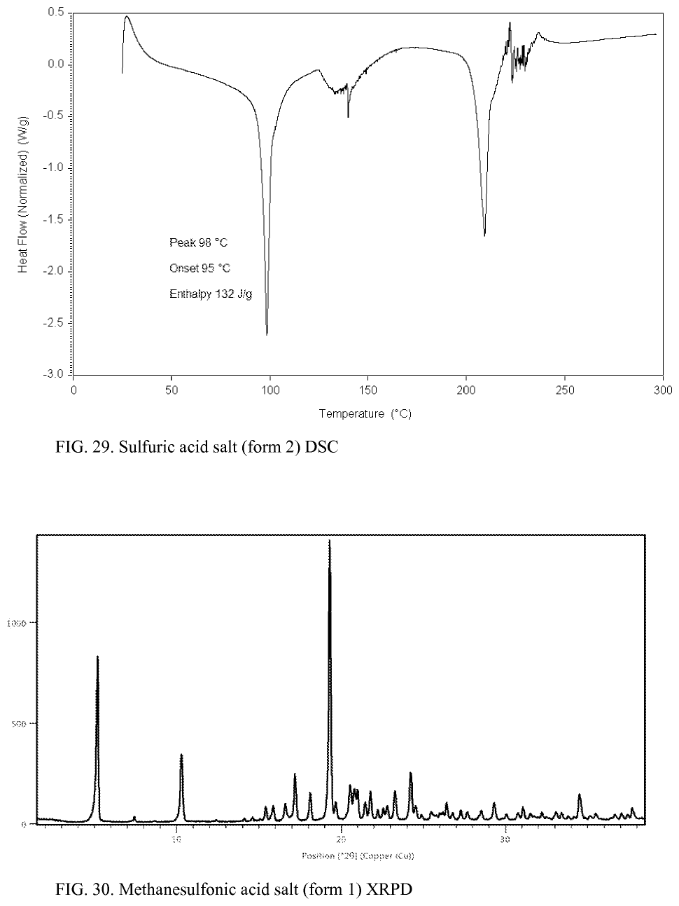

Sulfuric acid (0.028 g, 0.239 mmol) in 0.2 ml of ethanol was added to a solution of 2-(isoindolin-2-ylmethyl)-5-((1-(methylsulfonyl)piperidin-4-yl)methoxy)-4H-pyran-4-one (I) (0.1 g, 0.239 mmol) in 0.8 ml of ethanol at refluxing temperature, and refluxed until all solids were dissolved. The mixture was stirred and allowed to cool to room temperature. The precipitated solid was filtered, washed twice with ethanol, and dried under vacuum at 40° C. for 16 h to yield sulfuric acid salt of compound (I) (0.016 g, 13%). The product was analysed by XRPD and was found to be crystalline form 2. The X-ray powder diffraction pattern of form 2 is depicted in FIG. 28 and the differential scanning calorimetry (DSC) thermogram in FIG. 29. 1H NMR (400 MHz, DMSO-d6): δ ppm 1.21-1.38 (m, 2 H), 1.86 (br s, 3 H), 2.73 (br d, 2 H), 2.86 (s, 3 H), 3.59 (br d, 2 H), 3.75 (d, 2 H), 4.60-4.80 (m, 4 H), 6.65 (s, 1 H), 7.39 (br d, 4 H), 8.24 (s, 1 H), 10.70-11.78 (m, 1 H).

TABLE 18
X-ray powder reflections (up to 40° 2θ) and intensities
(normalized) of sulfuric acid salt crystalline form 2. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 5.10 | 17.32 | 17 |
| 7.44 | 11.88 | 5 |
| 8.64 | 10.23 | 5 |
| 10.23 | 8.64 | 5 |
| 12.34 | 7.17 | 3 |
| 14.18 | 6.24 | 2 |
| 14.52 | 6.09 | 4 |
| 14.89 | 5.94 | 2 |
| 15.88 | 5.58 | 25 |
| 16.51 | 5.36 | 4 |
| 17.05 | 5.20 | 7 |
| 17.14 | 5.17 | 9 |
| 17.27 | 5.13 | 8 |
| 17.98 | 4.93 | 11 |
| 19.16 | 4.63 | 100 |
| 19.55 | 4.54 | 4 |
| 20.42 | 4.35 | 11 |
| 20.71 | 4.29 | 26 |
| 20.94 | 4.24 | 10 |
| 21.28 | 4.17 | 6 |
| 21.59 | 4.11 | 14 |
| 22.33 | 3.98 | 17 |
| 22.68 | 3.92 | 12 |
| 23.30 | 3.82 | 61 |
| 24.03 | 3.70 | 13 |
| 24.53 | 3.63 | 31 |
| 25.57 | 3.48 | 3 |
| 26.19 | 3.40 | 18 |
| 26.79 | 3.33 | 2 |
| 27.13 | 3.28 | 5 |
| 27.43 | 3.25 | 8 |
| 28.00 | 3.18 | 6 |
| 28.38 | 3.14 | 6 |
| 28.53 | 3.13 | 5 |
| 29.00 | 3.08 | 6 |
| 29.91 | 2.98 | 6 |
| 30.51 | 2.93 | 3 |
| 30.80 | 2.90 | 6 |
| 31.39 | 2.85 | 8 |
| 31.96 | 2.80 | 5 |
| 32.70 | 2.74 | 4 |
| 32.96 | 2.72 | 7 |
| 33.36 | 2.68 | 3 |
| 34.17 | 2.62 | 17 |
| 35.32 | 2.54 | 6 |
| 36.40 | 2.47 | 5 |
| 36.68 | 2.45 | 7 |
| 37.41 | 2.40 | 4 |

### EXAMPLE 20

### Methanesulfonic Acid Salt Crystalline Form 1

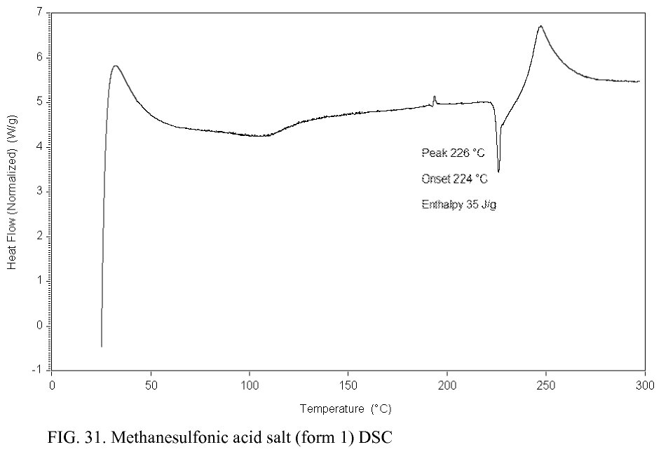

Methanesulfonic acid (0.919 g, 9.56 mmol) in 5 ml of ethanol was added to a solution of 2-(isoindolin-2-ylmethyl)-5-((1-(methylsulfonyl)piperidin-4-yl)methoxy)-4H-pyran-4-one (I) (4 g, 9.56 mmol) in 20 ml of ethanol at refluxing temperature. The solvent was removed in vacuo, followed by recrystallization (ACN:water 125:6, 26.2 ml) to yield methanesulfonic acid salt of compound (I) (3.01 g, 61%). The product was analysed by XRPD and was found to be crystalline form 1. The X-ray powder diffraction pattern of form 1 is depicted in FIG. 30 and the differential scanning calorimetry (DSC) thermogram in FIG. 31. 1H NMR (400 MHz, DMSO-d6): δ ppm 1.25-1.36 (m, 2 H), 1.78-1.92 (m, 3 H), 2.30 (s, 3 H), 2.67-2.78 (m, 2 H), 2.87 (s, 3 H), 3.52-3.62 (m, 2 H), 3.68-3.78 (m, 2 H), 4.60-4.84 (m, 4 H), 6.55-6.72 (m, 1 H), 7.28-7.50 (m, 4 H), 8.14-8.28 (m, 1 H), 10.57-11.62 (m, 1 H).

TABLE 19
X-ray powder reflections (up to 40° 2θ) and intensities
(normalized) of methanesulfonic acid salt crystalline form 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 5.16 | 17.12 | 49 |
| 7.41 | 11.93 | 2 |
| 8.63 | 10.24 | 0 |
| 10.28 | 8.60 | 22 |
| 12.38 | 7.14 | 0 |
| 14.10 | 6.28 | 1 |
| 14.60 | 6.06 | 1 |
| 15.41 | 5.75 | 5 |
| 15.83 | 5.59 | 4 |
| 16.60 | 5.34 | 6 |
| 17.18 | 5.16 | 15 |
| 18.11 | 4.90 | 10 |
| 19.29 | 4.60 | 100 |
| 19.67 | 4.51 | 5 |
| 20.53 | 4.32 | 12 |
| 20.80 | 4.27 | 9 |
| 20.99 | 4.23 | 10 |
| 21.45 | 4.14 | 6 |
| 21.77 | 4.08 | 10 |
| 22.22 | 4.00 | 4 |
| 22.55 | 3.94 | 4 |
| 22.79 | 3.90 | 5 |
| 23.26 | 3.82 | 10 |
| 24.22 | 3.67 | 18 |
| 24.52 | 3.63 | 4 |
| 24.87 | 3.58 | 2 |
| 25.48 | 3.49 | 2 |
| 26.04 | 3.42 | 2 |
| 26.40 | 3.37 | 6 |
| 26.77 | 3.33 | 2 |
| 27.27 | 3.27 | 4 |
| 27.65 | 3.22 | 3 |
| 28.48 | 3.13 | 3 |
| 29.29 | 3.05 | 6 |
| 30.04 | 2.97 | 2 |
| 30.71 | 2.91 | 2 |
| 30.79 | 2.90 | 2 |
| 31.04 | 2.88 | 4 |
| 31.51 | 2.84 | 2 |
| 32.20 | 2.78 | 2 |
| 32.81 | 2.73 | 0 |
| 33.05 | 2.71 | 2 |
| 33.39 | 2.68 | 3 |
| 33.80 | 2.65 | 1 |
| 34.14 | 2.62 | 1 |
| 34.49 | 2.60 | 10 |
| 35.12 | 2.55 | 1 |
| 35.47 | 2.53 | 2 |
| 36.64 | 2.45 | 2 |
| 37.04 | 2.43 | 2 |
| 37.41 | 2.40 | 2 |
| 37.70 | 2.38 | 5 |

### EXAMPLE 21

### Single Crystal X-ray Diffraction Data of p-toluenesulfonic Acid Salt

Unit cell parameters of crystalline p-toluenesulfonic acid salt of compound (I) were determined from single crystal X-ray diffraction data and are summarized below, T=293(2) K, radiation wavelength CuKα (λ=1.5418 Å), crystal size 0.3×0.3×0.05 mm³, structural formula C₂₈H₃₄N₂O₈S₂. As the crystalline form was determined to be a variable hydrate, a unit cell volume expansion up to about 4% is found when water is included.

| | |
|---|---|
| Crystal system | Monoclinic |
| Space group | P2₁ |

| | | |
|---|---|---|
| Unit cell dimensions | a = 6.02369(10) Å | α = 90° |
| | b = 12.21533(19) Å | β = 92.5099(16)° |
| | c = 20.1514(4) Å | γ = 90° |
| Volume | V = 1481.35(4) Å³ | |

| | |
|---|---|
| Z | 2 |
| Goodness-of-fit | 1.030 |
| R factor | 0.0657 |
| Morphology | Prismatic |

### EXAMPLE 22

### Melting Points of the Salts of Compound (I)

Melting point of each salt of compound (I) was determined by observing the phase change during hot stage microscopy in open chamber. Heating rate 10° C./min. The results are shown in Table 20. A crystalline solid with high melting point tends to be easy to purify by re-crystallisation and stable on storage.

TABLE 20
Melting points of the salts of compound
(I) observed by hot stage microscopy

| Salt form of compound (I) | Melting point |
|---|---|
| 1,5-Naphthalenedisulfonic acid | 278° C. |
| 2-Naphthalenesulphonic acid | 255° C. |
| p-Toluenesulfonic acid salt | 255° C. * |
| Hydrobromic acid salt | 221° C. |
| Nitric acid salt | 199° C. |
| Benzenesulfonic acid salt | 210° C. |
| Hydrochloric acid salt (form 3) | 190° C. |
| Maleic acid salt (form 3) | 170° C. |
| | (decomposition) |
| 1,2-Ethanedisulfonic acid salt | 133° C. |
| | (darkening at 75° C.) |
| Oxalic acid salt (form 1) | 170° C. |
| Ethanesulfonic acid salt | 190° C. |
| Sulfuric acid salt (form 2) | 100° C. |
| Methanesulfonic acid salt | 190° C. |
| | (darkening at 65° C.) |

* melting point from DSC

### EXAMPLE 23

### Weight Loss of the Salts of Compound (I) During Heating

Weight loss of each salt of compound (I) during heating was determined by thermogravimetric analysis (TGA) thermograms collected on TGA equipment (TA Instruments). Heating rate 10° C./min, 25-300° C., open pan. The results are shown in Table 21.

TABLE 21
Weight loss during heating of the salts of compound (I)

| Salt form of compound (I) | Weight loss | |
|---|---|---|
| 1,5-Naphthalenedisulfonic acid | 0.3% | (25-220° C.) |
| 2-Naphthalenesulphonic acid | <0.1% | (25-225° C.) |
| p-Toluenesulfonic acid salt | 0.2% | (30-200° C.) * |
| Hydrobromic acid salt | 0.1% | (25-220° C.) |
| Nitric acid salt | 0.9% | (25-205° C.) |
| Benzenesulfonic acid salt | 0.3% | (25-220° C.) |
| Hydrochloric acid salt (form 3) | 0.5% | (25-190° C.) |
| Maleic acid salt (form 3) | 3.2% | (25-120° C.) |
| 1,2-Ethanedisulfonic acid salt | 9.9% | (25-100° C.) |
| Oxalic acid salt (form 1) | 0.4% | (25-175° C.) |
| Ethanesulfonic acid salt | <0.3% | (25-190° C.) |
| Sulfuric acid salt (form 2) | 8% | (25-100° C.) |
| Methanesulfonic acid salt | 5% | (25-75° C.) |

* Method: heating rate 10° C./min, 30-250° C., open pan

### EXAMPLE 24

### Chemical Stability of the Salts

The degradation rate of various salts of compound (I) was determined by measuring the amount of shelf-life determining degradation product 2-[(5-{[1-(methanesulfonyl)piperidin-4-yl]methoxy}-4-oxo-4H-pyran-2-yl)methyl]-2,3-dihydro-1H-isoindol-1 -one (degradation product A) from an accelerated stability study wherein samples were stored at various different temperature and relative humidity conditions. Each sample consisted of a pressed tablet containing a constant amount of the tested salt together with conventional excipients. The predicted increase (Δ %) of the degradation product A after 5 years was calculated with ASAPprime® software using the following parameters:
Bottle volume and material: 100 ml (HDPE HIS)
Desiccant: Silica (1 g)
Tablet amount: 60
Conditions: 25° C./60% RH

The predicted increase (Δ %) of degradation product A after 5 years for each tested salt of compound (I) is shown in Table 22.

TABLE 22
Predicted increase of degradation product A after 5 years

| | Increase (Δ%) of the degradation |
|---|---|
| Salt form of compound (I) | product A after 5 years |
| 1,5-Naphthalenedisulfonic acid | 0.01 |
| 2-Naphthalenesulphonic acid | 0.03 |
| p-Toluenesulfonic acid salt | 0.07 |
| Hydrobromic acid salt | 0.11 |
| Nitric acid salt | 0.19 |
| Benzenesulfonic acid salt | 0.28 |
| Hydrochloric acid salt (form 3) | 0.34 |
| Maleic acid salt (form 3) | 0.39 |
| 1,2-Ethanedisulfonic acid salt | 0.60 |
| Oxalic acid salt (form 1) | 3.41 |
| Ethanesulfonic acid salt | 4.53 |
| Sulfuric acid salt (form 2) | 7.38* |
| Methanesulfonic acid salt | 10.05* |

*Non-Arrhenius behaviour observed

1. A salt of 2-(isoindolin-2-ylmethyl)-5-((1-(methylsulfonyl)piperidin-4-yl)-methoxy)-4H-pyran-4-one (I) with an acid selected from the group consisting of
p-toluenesulfonic acid,
2-naphthalenesulfonic acid,
1,5-naphthalenedisulfonic acid,
hydrobromic acid,
nitric acid,
benzenesulfonic acid,
hydrochloride acid,
maleic acid,
1,2-ethanedisulfonic acid,
oxalic acid,
ethanesulfonic acid,
sulfuric acid and
methanesulfonic acid.

2. The salt according to claim 1, which is a salt of 2-(isoindolin-2-ylmethyl)-5-((1-(methylsulfonyl)piperidin-4-yl)methoxy)-4H-pyran-4-one (I) with an acid selected from the group consisting of
p-toluenesulfonic acid,
2-naphthalenesulfonic acid,
1,5-naphthalenedisulfonic acid and
hydrobromic acid.

3. The salt according to claim 1 which is crystalline.

4. The salt according to claim 2, which is a crystalline p-toluenesulfonic acid salt of 2-(isoindolin-2-ylmethyl)-5-((1-(methylsulfonyl)piperidin-4-yl)-methoxy)-4H-pyran-4-one (I).

5. The salt according to claim 4, which is of crystalline form 1 having an X-ray powder diffraction pattern comprising peaks, expressed in degrees 2-theta (±0.2), at 4.4, 15.2, 18.4, 19.1, 20.8 and 22.4.

6. The salt according to claim 5, wherein the crystalline form 1 has an X-ray powder diffraction pattern comprising y peaks, expressed in degrees 2-theta (±0.2), at 4.4, 8.8, 11.4, 15.2, 16.5, 17.1, 18.4, 19.1, 20.8 and 22.4.

7. The salt according to claim 4, wherein the crystalline form 1 has the following unit cell parameters at T=293(2) K:

| | |
|---|---|
| Crystal system | Monoclinic |
| Space group | P2₁ |
| | | |
|---|---|---|
| Unit cell dimensions | a = 6.02369(10) Å | α = 90° |
| | b = 12.21533(19) Å | β = 92.5099(16)° |
| | c = 20.1514(4) Å | γ = 90° |
| Volume | V = 1481.35(4) Å³ | |
| | |
|---|---|
| Z | 2 |
| Goodness-of-fit | 1.030 |
| R factor | 0.0657 |
| Morphology | Prismatic |

8. The salt according to claim 2, which is a crystalline 2-naphthalenesulfonic acid salt of 2-(isoindolin-2-ylmethyl)-5-((1-(methylsulfonyl)piperidin-4-yl)-methoxy)-4H-pyran-4-one (I).

9. The salt according to claim 8, which is of crystalline form 1 having an X-ray powder diffraction pattern comprising peaks, expressed in degrees 2-theta (±0.2), at 4.3, 8.7, 13.0, 18.8 and 27.1.

10. The salt according to claim 9, wherein the crystalline form 1 has an X-ray powder diffraction pattern comprising peaks, expressed in degrees 2-theta (±0.2), at 4.3, 8.7, 13.0, 18.8, 21.7, 27.1 and 35.8.

11. The salt according to claim 2, which is a crystalline 1,5-naphthalenedisulfonic acid salt of 2-(isoindolin-2-ylmethyl)-5-((1-(methylsulfonyl)piperidin-4-yl)methoxy)-4H-pyran-4-one (I).

12. The salt according to claim 11, which is of crystalline form 1 having an X-ray powder diffraction pattern comprising peaks, expressed in degrees 2-theta (±0.2), at 10.6, 17.6, 20.2, 20.4, 22.8 and 24.8.

13. The salt according to claim 12, wherein the crystalline form 1 has an X-ray powder diffraction pattern comprising peaks, expressed in degrees 2-theta (±0.2), at 5.9, 9.2, 10.6, 15.5, 17.1, 17.6, 20.2, 20.4, 22.8 and 24.8.

14. The salt according to claim 2, which is a crystalline hydrobromic acid salt of 2-(isoindolin-2-ylmethyl)-5-((1-(methylsulfonyl)piperidin-4-yl)-methoxy)-4H-pyran-4-one (I).

15. The salt according to claim 14, which is of crystalline form 1 having an X-ray powder diffraction pattern comprising peaks, expressed in degrees 2-theta (±0.2), at 5.3, 10.5, 13.6, 18.3, 21.4 and 26.9.

16. The salt according to claim 15, wherein the crystalline form 1 has an X-ray powder diffraction pattern comprising peaks, expressed in degrees 2-theta (±0.2), at about 5.3, 10.5, 13.6, 16.9, 18.3, 18.8, 21.4, 22.6 and 26.9.

17. A method of preparing a crystalline salt according to claim 4, comprising dissolving compound (I) and p-toluenesulfonic acid in a mixture of acetonitrile and water, cooling the mixture and isolating the crystalline product.

18. A method of preparing a crystalline salt according to claim 8, comprising dissolving compound (I) and 2-naphthalenesulfonic acid in ethanol or a mixture of ethanol and water, cooling the mixture and isolating the crystalline product.

19. A method of preparing a crystalline salt according to claim 11, comprising dissolving compound (I) and 1,5-naphthalenedisulfonic acid in ethanol, a mixture of ethanol and water or a mixture of acetonitrile and water, cooling the mixture and isolating the crystalline product.

20. A method of preparing a crystalline salt according to claim 14, comprising dissolving compound (I) and hydrobromic acid in ethanol, or a mixture of water with ethanol or isopropanol, cooling the mixture and isolating the crystalline product.

21. A pharmaceutical composition comprising the salt according to claim 1 as an active ingredient together with one or more excipients.

22. The pharmaceutical composition according to claim 21, which is in the form of a tablet, capsule, granule, powder or suspension.

23. The pharmaceutical composition according to claim 22, which is in the form of a tablet or capsule.

24. The pharmaceutical composition according to claim 23, which is in the form of a tablet.

25. A method for the treatment of a hormonally regulated cancer, wherein the method comprises administering a therapeutically effective amount of the salt according to claim 1.

26. The method of claim 25, wherein the cancer is prostate cancer or breast cancer.
